SEQUENCE LISTING (49 sequences)

SEQ ID NO: 1 - DNA, 494 bp, Artificial Sequence
  chemically synthesized
atggaatgcacctgggtttttctcttcctcctgtcagtaattgcaggtgtccaatcccaggttcatctgcagcagtctggggctgagctggtgaggcctggggcttcagtgacgccgccctgcaaggcttcgggctacacatttactgactatgaaatgcactgggtgaaacagacacctgtgcatggcctggaatggattggcgctattgatcctgaaactggtgctattgtctacaatcagaagttcaagggcaaggccacactgactgcagacaaatcctccaacacagcccacatggagctccgcagcctgacatctgaggactctgccgtctattactgtacaagagagtttacttactggggccaagggactctggtcactgtctctgcagccaaaacgacacccccatccgtcttccccctggcacaagccaaattctgcagatatccatcacactggcggccgctcgagcatctattctttgggaa

SEQ ID NO: 2 - protein, 164 aa, Artificial Sequence
  chemically synthesized
MECTWVFLFLLSVIAGVQSQVHLQQSGAELVRPGASVTPPCKASGYTFTDYEMHWVKQTPVHGLEWIGAIDPETGAIVYNQKFKGKATLTADKSSNTAHMELRSLTSEDSAVYYCTREFTYWGQGTLVTVSAAKTTPPSVFPLAQAKFCRYPSHWRPLEHLFFG

SEQ ID NO: 3 - DNA, 447 bp, Artificial Sequence
  chemically synthesized
atggaatgcacctgggttattctcttcctcctgtcagtaattgcaggtgtccaatcccaggttcatctgcagcagtctggggctgagctggtgaggcctggggcttcagtgacgctgccctgcaaggcttcgggctacacatttactgactatgaaatgcactgggtgaaacagacacctgtgcatggcctggaatggatcggcgctattgatcctgaaactggtgctattgtctacaatcagaagctcaagggcaaggccacactggctgcagacaaatcctccaacacagcctacatggagctccgcagcctgacatctgaggactctgccgtctattactgtacaagagagtttacttactggggccaagggactctggtcactgtctctgcagccaaaacgacacccccacccgtttatccactggccccctggaagcttggg

SEQ ID NO: 4 - protein, 149 aa, Artificial Sequence
  chemically synthesized
MECTWVILFLLSVIAGVQSQVHLQQSGAELVRPGASVTLPCKASGYTFTDYEMHWVKQTPVHGLEWIGAIDPETGAIVYNQKLKGKATLAADKSSNTAYMELRSLTSEDSAVYYCTREFTYWGQGTLVTVSAAKTTPPPVYPLAPWKLG

SEQ ID NO: 5 - DNA, 446 bp, Artificial Sequence
  chemically synthesized
atgaaatggacctgggttttcctcttcctcctgtcagtaattgcaggtgtccaatcccaggttcatctgcagcagtctggggctgagctggtgaggcctggggcttcagtgacgctgccctgcaaggcttcgggctacacatttactgactatgaaatgcactgggtgaaacagacacctgtgcatggcctggaatggattggcgctattgatcctgaaactggtgctattgtctacaatcagaagttcaagggcaaggccacactgactgcagacaaatcctccaacacagcctacatggagctccgcagcctgacatctgaggactctgccgtctattactgtacaagagagtttacttactggggccaagggactctggtcactgtctctgcagccaaaacgacacccccatccgtttatccactggcccctggaagcttggg

SEQ ID NO: 6 - protein, 148 aa, Artificial Sequence
  chemically synthesized
MKWTWVFLFLLSVIAGVQSQVHLQQSGAELVRPGASVTLPCKASGYTFTDYEMHWVKQTPVHGLEWIGAIDPETGAIVYNQKFKGKATLTADKSSNTAYMELRSLTSEDSAVYYCTREFTYWGQGTLVTVSAAKTTPPSVYPLAPGSL

SEQ ID NO: 7 - DNA, 446 bp, Artificial Sequence
  chemically synthesized
atggaatgcagcagggttattctcttcctcctgtcagtaattgcaggtgtccaatcccaggttcatctgcagcagtctggggctgagctggtgaggcctggggcttcagtgacgctgccctgcaaggcttcgggctacacatttactgactatgaaatgcactgggtgaaacagacacctgtgcatggcctggaatggattggcgctattgatcctgaaactggtgctattgtctacaatcagaagttcaagggcaaggccacactgactgcagacaaatcctccaacacagcctacatggagctccgcagcctgacatctgaggactctgccgtctattactgtacaagagagtttacttactggggccaagggactctggtcactgtctctgcagccaaaacgacacccccacccgtctatcccctggcccctggaagcttggg

SEQ ID NO: 8 - protein, 148 aa, Artificial Sequence
  chemically synthesized
MECSRVILFLLSVIAGVQSQVHLQQSGAELVRPGASVTLPCKASGYTFTDYEMHWVKQTPVHGLEWIGAIDPETGAIVYNQKFKGKATLTADKSSNTAYMELRSLTSEDSAVYYCTREFTYWGQGTLVTVSAAKTTPPPVYPLAPGSL

SEQ ID NO: 9 - protein, 148 aa, Artificial Sequence
  chemically synthesized
MECTWVILFLLSVIAGVQSQVHLQQSGAELVRPGASVTLPCKASGYTFTDYEMHWVKQTPVHGLEWIGAIDPETGAIVYNQKFKGKATLTADKSSNTAYMELRSLTSEDSAVYYCTREFTYWGQGTLVTVSAAKTTPPSVYPLAPGSL

SEQ ID NO: 10 - protein, 8 aa, Artificial Sequence
  chemically synthesized
GYTFTDYE

SEQ ID NO: 11 - protein, 8 aa, Artificial Sequence
  chemically synthesized
IDPETGAI

SEQ ID NO: 12 - protein, 6 aa, Artificial Sequence
  chemically synthesized
TREFTY

SEQ ID NO: 13 - DNA, 357 bp, Artificial Sequence
  chemically synthesized
tctcctttcaacaaagcacatttcgatttccagcttggtgcctccaccgaacgtccacggaaaatgtgtaccttgcacgcagtaataaactcccaaatcctcagcctccactctgctgattttcagtgaaaaatctgttcctgaaccagtgccagtgaacctgtcagggactccagagttcagtttagacaccagatagattaggcgctttggagactggcctggcctctgaaataaccaattcaagtaggtttttccattactaaataagaggctctgacttgacctgcaagagacagaggctggttgtccaatggtaaccgacaaagtgagtggagtttgggtcatcaaaacatc

SEQ ID NO: 14 - protein, 119 aa, Artificial Sequence
  chemically synthesized
DVLMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIEMCFVERR

SEQ ID NO: 15 - DNA, 367 bp, Artificial Sequence
  chemically synthesized
attggatatcctcgcagcatctcggcttgatgttttgatgacccaaactccactcactttgtcggttaccattggacaaccagcctctgtctcttgcaggtcaagtcagagcctcttatttagtaatggaaaaacctatttgaattggttatttcagaggccaggccagtctccaaagcgcctaatctatctggtgtctaaactgaactctggagtccctgacaggttcactggcactggttcaggaacagatttttcactgaaaatcagcagagtggaggctgaggatttgggagtttattactgcgtgcaaggtacacattttccgtggacgttcggtggaggcaccaagctggaaatcaaacgg

SEQ ID NO: 16 - protein, 122 aa, Artificial Sequence
  chemically synthesized
LDILAASRLDVLMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIKR

SEQ ID NO: 17 - DNA, 374 bp, Artificial Sequence
  chemically synthesized
ctttcgcgatgatccttgcacgcatttcaggcttggatgttttgatgacccaaactccactcactttgtcggttaccattggacaaccagcctctgtctcttgcaggtcaagtcagagcctcttatttagtaatggaaaaacctatttgaattggttatttcagaggccaggccagtctccaaagcgcctaatctgtctggtgtctaaactgaactctggagtccctgacaggttcactggcactggttcaggaacagatttttcactgaaaatcagcagagtggaggctgaggatttgggattttattactgcgtgcaaggtacacattttccgtggacgttcggtggaggcaccaagctggaaatcaaacgg

SEQ ID NO: 18 - protein, 124 aa, Artificial Sequence
  chemically synthesized
FAMILARISGLDVLMTQTPLTLSVTIGQPASVSCRSSQSLLFSNGKTYLNWLFQRPGQSPKRLICLVSKLNSGVPDRFTGTGSGTDFSLKISRVEAEDLGFYYCVQGTHFPWTFGGGTKLEIKR

SEQ ID NO: 19 - DNA, 391 bp, Artificial Sequence
  chemically synthesized
gatgttttgatgacccaaactccactcactttgtcggttaccattggacaaccagcctctgtctcttgcaggtcaagtcagagcctcttatttagtaatggaaaaacctatttgaattggttatttcagaggccaggccagtctccaaagcgcctaatctatctggtgtctaaactgaactctggagtctctgacaggttcactggcactggttcagaaacagatttttcactgaaaatcagcagagtggaggctgaggatttgggagtttattactgcgtgcaaggtacatattttccgtggacgttcggtggaggcaccaagctggaaatcaaacggccctttttaattctgcagatatcctatcacaacgttgctggccgcggccgct

SEQ ID NO: 20 - protein, 130 aa, Artificial Sequence
  chemically synthesized
DVLMTQTPLTLSVTIGQPASVSCRSSQSLLFSNGKTYLNWLFQRPGQSPKRLIYLVSKLNSGVSDRFTGTGSETDFSLKISRVEAEDLGVYYCVQGTYFPWTFGGGTKLEIKRPFLILQISYHNVAGRGR

SEQ ID NO: 21 - DNA, 343 bp, Artificial Sequence
  chemically synthesized
gatgttttgatgacccaaactccactcactttgtcggttaccattggacaaccagcctctgtctcttgcaggtcaagtcagagcctcttatttagtaatggaaaaacctatttgaattggttatttcagaggccaggccagtctccaaagcgcctaatctatctggtgtctaaactgaactctggagtccctgacaggttcaccggcactggttcaggaacagattttccactgaaaatcagcagagtggaggctgaggatttgggagtttattactgcgtgcaaggtacacattttccgtggacgttcggtgccctttttaaaggaggccgtgataaaaaat

SEQ ID NO: 22 - protein, 114 aa, Artificial Sequence
  chemically synthesized
DVLMTQTPLTLSVTIGQPASVSCRSSQSLLFSNGKTYLNWLFQRPGQSPKRLIYLVSKLNSGVPDRFTGTGSGTDFPLKISRVEAEDLGVYYCVQGTHFPWTFGALFKGGRDKK

SEQ ID NO: 23 - protein, 113 aa, Artificial Sequence
  chemically synthesized
DVLMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIKR

SEQ ID NO: 24 - protein, 11 aa, Artificial Sequence
  chemically synthesized
QSLLFSNGKTY

SEQ ID NO: 25 - protein, 3 aa, Artificial Sequence
  chemically synthesized
LVS

SEQ ID NO: 26 - protein, 9 aa, Artificial Sequence
  chemically synthesized
VQGTHFPWT

SEQ ID NO: 27 - DNA, 433 bp, Artificial Sequence
  chemically synthesized
atgaaatgcagctgggttttcctcttcctcctgtcagtaattgcaggtgtccaatcccgggttcaactgcagcagtctgggtctgagctggtgaggcctggggcttcagtgacgctgtcctgcaaggcttcgggctacacatttactgactatgaaatgcactgggtgaagcagacacctgtgcatggcctggaatggattggagcgattgatcctgaaactggtaaaactgcctacaatcagaagttcaagggcaagaccacactgactgcagacaaatcctccagcacagcctacatggagttccgcagcctgacatctgaggactctgccgtccattactgtacaagagagttgacttactggggccaagggactctggtcactgtctctgcagccaaaacgacacccccatcggtcttccccctggcac

SEQ ID NO: 28 - protein, 144 aa, Artificial Sequence
  chemically synthesized
MKCSWVFLFLLSVIAGVQSRVQLQQSGSELVRPGASVTLSCKASGYTFTDYEMHWVKQTPVHGLEWIGAIDPETGKTAYNQKFKGKTTLTADKSSSTAYMEFRSLTSEDSAVHYCTRELTYWGQGTLVTVSAAKTTPPSVFPLA

SEQ ID NO: 29 - DNA, 433 bp, Artificial Sequence
  chemically synthesized
atggaatgcacctgggttttcctcttcctcctgtcagtaattgcaggtgtccaatcccaggttcaactgcagcagtctgggtctgagctggtgaggcctggggcttcagtgacgctgtcctgcaaggcttcgggctacacatttactgactatgaaatgcactgggtggagcagacacctgtgcatggcctggaatggattggagcgattgatcctgaaactggtaaaactgcctacaatcagaagttcaagggcaagaccacactgactgcagacaaatcctccagcacagcctacatggagttccgcagcctgacatctgaggactctgccgtccattactgtacaagagagttgacttactggggccaagggactctggtcactgtctctgcagccaaaacgacacccccatccgtcttccccctggcac

SEQ ID NO: 30 - protein, 144 aa, Artificial Sequence
  chemically synthesized
MECTWVFLFLLSVIAGVQSQVQLQQSGSELVRPGASVTLSCKASGYTFTDYEMHWVEQTPVHGLEWIGAIDPETGKTAYNQKFKGKTTLTADKSSSTAYMEFRSLTSEDSAVHYCTRELTYWGQGTLVTVSAAKTTPPSVFPLA

SEQ ID NO: 31 - DNA, 433 bp, Artificial Sequence
  chemically synthesized
atgaaatgcagctgggttttcctcttcctcctgtcagtaattgcaggtgtccaatcccaggttcaactgcagcagtctgggtctgagctggtgaggcctggggcttcagtgacgctgtcctgcaaggcttcgggctacacatttactgactatgaaatgcactgggtgaagcagacacctgtgcatggcctggaatggattggagcgattgatcctgaaactggtaaaactgcctacaatcagaagttcaagggcaagaccacactgactgcagacaaatcccccagcacagcctacatggagttccgcagcctgacatctgaggactctgccgtccattactgtacaagagagttgacttactggggccaagggactctggtcactgtctctgcagccaaaacgacacccccatcggtcttccccctggcac

SEQ ID NO: 32 - protein, 144 aa, Artificial Sequence
  chemically synthesized
MKCSWVFLFLLSVIAGVQSQVQLQQSGSELVRPGASVTLSCKASGYTFTDYEMHWVKQTPVHGLEWIGAIDPETGKTAYNQKFKGKTTLTADKSPSTAYMEFRSLTSEDSAVHYCTRELTYWGQGTLVTVSAAKTTPPSVFPLA

SEQ ID NO: 33 - DNA, 446 bp, Artificial Sequence
  chemically synthesized
atgaaatgcagctgggttttcctcttcctcctgtcagtaattgcaggtgtccaatcccaggttcaactgcagcagtctgggtctgagctggtgaggcctggggcttcagtgacgctgtcctgcaaggcttcgggctacacatttactgactatgaaatgcactgggtgaagcagacacctgtgcatggcctggaatggattggagcgattgatcctgaaactggtaaaactgcctacaatcagaagttcaagggcaagaccacactgactgcagacaaatcctccagcacagcctacatggagttccgcagcctgacatctgaggactctgccgtccattactgcacaagagagttgacttactggggccaagggactctggtcactgtctctgcagccaaaacgacacccccacccgtctatccattggcccctggaagcttggg

SEQ ID NO: 34 - protein, 148 aa, Artificial Sequence
  chemically synthesized
MKCSWVFLFLLSVIAGVQSQVQLQQSGSELVRPGASVTLSCKASGYTFTDYEMHWVKQTPVHGLEWIGAIDPETGKTAYNQKFKGKTTLTADKSSSTAYMEFRSLTSEDSAVHYCTRELTYWGQGTLVTVSAAKTTPPPVYPLAPGSL

SEQ ID NO: 35 - DNA, 446 bp, Artificial Sequence
  chemically synthesized
atgaaatggacctgggtttttctcttcctcctgtcagtaattgcaggtgtccaatcccaggttcaactgcagcagtctgggtctgagctggtgaggcctggggcttcagtgacgctgtcctgcaaggcttcgggctacacatttactgactatgaaatgcactgggtgaagcagacacctgtgcatggcctggaatggattggagcgattgatcctgaaactggtaaaactgcctacaatcagaagttcaagggcaagaccacactgactgcagacaaatcctccagcacagcctacatggagttccgcagcctgacatctgaggactctgccgtccattactgtacaagagagttgacttactggggccaagggactctggtcactgtctctgcagccaaaacgacacccccacccgtctatcccctggcccctggaagcttggg

SEQ ID NO: 36 - protein, 148 aa, Artificial Sequence
  chemically synthesized
MKWTWVFLFLLSVIAGVQSQVQLQQSGSELVRPGASVTLSCKASGYTFTDYEMHWVKQTPVHGLEWIGAIDPETGKTAYNQKFKGKTTLTADKSSSTAYMEFRSLTSEDSAVHYCTRELTYWGQGTLVTVSAAKTTPPPVYPLAPGSL

SEQ ID NO: 37 - DNA, 447 bp, Artificial Sequence
  chemically synthesized
atggaatggagctgggttttcctcttcctcctgtcagtaattgcaggtgtccaatcccaggttcaactgcagcagtctgggtctgagctggtgaggcctggggcttcagtgacgctgtcctgcaaggcttcgggctacacatttactgactatgaaatgcactgggtgaagcagacacctgtgcatggcctggaatggataggagcgattgatcctgaaactggtaaaactgcctacaatcagaagttcaagggcaagaccacactgactgcagacaaatcctccagcacagcctacatggagttccgcagcctgacatctgaggactctgccgtccattactgtacaagagagttgacttactggggccaagggactctggtcactgtctctgcagccaaaacgacacccccacccgtctatccattggccccctggaagcttggg

SEQ ID NO: 38 - protein, 149 aa, Artificial Sequence
  chemically synthesized
MEWSWVFLFLLSVIAGVQSQVQLQQSGSELVRPGASVTLSCKASGYTFTDYEMHWVKQTPVHGLEWIGAIDPETGKTAYNQKFKGKTTLTADKSSSTAYMEFRSLTSEDSAVHYCTRELTYWGQGTLVTVSAAKTTPPPVYPLAPWKLG

SEQ ID NO: 39 - protein, 145 aa, Artificial Sequence
  chemically synthesized
MKCSWVFLFLLSVIAGVQSQVQLQQSGSELVRPGASVTLSCKASGYTFTDYEMHWVKQTPVHGLEWIGAIDPETGKTAYNQKFKGKTTLTADKSSSTAYMEFRSLTSEDSAVHYCTRELTYWGQGTLVTVSAAKTTPPSVFPLAP

SEQ ID NO: 40 - protein, 8 aa, Artificial Sequence
  chemically synthesized
IDPETGKT

SEQ ID NO: 41 - protein, 6 aa, Artificial Sequence
  chemically synthesized
TRELTY

SEQ ID NO: 42 - DNA, 499 bp, Artificial Sequence
  chemically synthesized
atgaagttgcctgttaggctgttggtgctgatgttctggattcccgcctcctcctccgacgtgctgatgacccagacccccctgaccctgtccgtgaccatcggccagcctgcctccgtgtcctgccggtcctcccagtccctgctgttctccaacggcaagacctacctgaactggctgttccagcggcctggccagtcccccaagcggctgatctacctggtgtccaagctgaactccggcgtgcccgaccggtttacaggcaccggctctggcaccgacttcagcctgaagatcagccgggtggaagccgaggacctgggcgtgtactactgcgtgcagggcacccacttcccttggaccttcggcggaggcaccaagctggaaatcaagcgggccgatgccgcccctaccgtgtccatcttcccaccctccagcgagcagctgacctctggcggcgcttccgtcgtgtgcttcctgaacaacttctaccccaaag

SEQ ID NO: 43 - protein, 166 aa, Artificial Sequence
  chemically synthesized
MKLPVRLLVLMFWIPASSSDVLMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIKRADAAPTVSIFPPSSEQLTSGGASVVCFLNNFYPK

SEQ ID NO: 44 - DNA, 500 bp, Artificial Sequence
  chemically synthesized
atgaagttgcctgttaggctgttggtgctgatgttctggattcccgcctcctcctccgacgtgctgatgacccagacccccctgaccctgtccgtgaccatcggccagcctgcctctgtgtcctgccggtcctcccagtccctgctgttctccaacggcaagacctacctgaactggctgttccagcggcctggccagtcccccaagcggctgatctacctggtgtccaagctgaactccggcgtgcccgaccggtttacaggcaccggctctggcaccgacttcagcctgaagatcagccgggtggaagccgaggacctgggcgtgtactactgcgtgcagggcacccacttcccttggaccttcggcggaggcaccaagctggaaatcaagcgggccgatgccgcccctaccgtgtccatcttcccaccctccagcgagcagctgacctctggcggcgcttccgtcgtgtgcttcctgaacaacttctaccccagaga

SEQ ID NO: 45 - protein, 166 aa, Artificial Sequence
  chemically synthesized
MKLPVRLLVLMFWIPASSSDVLMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIKRADAAPTVSIFPPSSEQLTSGGASVVCFLNNFYPR

SEQ ID NO: 46 - DNA, 499 bp, Artificial Sequence
  chemically synthesized
atgaagttgcctgtaggctgttggtgctgatgttctggattcccgcctcctcctccgacgtgcagatgacccagacccccctgaccctgtccgtgaccatcggccagcctgcctctgtgtcctgccggtcctcccagtccctgctgttctccaacggcaagacctacctgaactggctgttccagcggcctggccagtcccccaagcggctgatctacctggtgtccaagctgaactccggcgtgcccgaccggtttacaggcaccggctctggcaccgacttcagcctgaagatcagccgggtggaagccgaggacctgggcgtgtactactgcgtgcagggcacccacttcccttggaccttcggcggaggcaccaagctggaaatcaagcgggccgatgccgcccctaccgtgtccatcttcccaccctccagcgagcagctgacctctggcggcgcttccgtcgtgtgcttcctgaacaacttctaccccagaga

SEQ ID NO: 47 - protein, 165 aa, Artificial Sequence
  chemically synthesized
EVACRLLVLMFWIPASSSDVQMTQTPLTLSVTIGQPASVSCRSSQSLLFSNGKTYLNWLFQRPGQSPKRLIYLVSKLNSGVPDRFTGTGSGTDFSLKISRVEAEDLGVYYCVQGTHFPWTFGGGTKLEIKRADAAPTVSIFPPSSEQLTSGGASVVCFLNNFYPR

SEQ ID NO: 48 - DNA, 500 bp, Artificial Sequence
  chemically synthesized
atgaagttgcctgttaggctgttggtgctgatgttctggattcccgcctcctcctccgacgtgctgatgacccagacccccctgaccctgtccgtgaccatcggccagcctgcctctgtgtcctgccggtcctcccagtccctgctgttctccaacggcaagacctacctgaactggctgttccagcggcctggccagtcccccaagcggctgatctacctggtgtccaagctgaactccggcgtgcccgaccggtttacaggcaccggctctggcaccgacttcagcctgaagatcagccgggtggaagccgaggacctgggcgtgtactactgcgtgcagggcacccacttcccttggaccttcggcggaggcaccaagctggaaatcaagcgggccgatgccgcccctaccgtgtccatcttcccaccctccagcgagcagctgacctctggcggcgcttccgtcgtgtgcttcctgaacaacttctaccccaaaga

SEQ ID NO: 49 - protein, 166 aa, Artificial Sequence; identical to SEQ ID NO: 45
  chemically synthesized

(19) United States
(12) Patent Application Publication
(10) Pub. No.: US 2012/0264710 A1
(43) Pub. Date: Oct. 18, 2012

(54) Systems and Methods for Detecting hENT1 Expression in Hematological Disorders

(76) Inventors: Marit Liland Sandvold, Oslo (NO); Birgitte Boonstra Booij, Nordby (NO); Tove Flem Jacobsen, Oslo (NO); Bruce H. Davis, Bangor, ME (US)

(21) Appl. No.: 13/446,004

(22) Filed: Apr. 13, 2012

Related U.S. Application Data
(60) Provisional application No. 61/475,953, filed on Apr. 15, 2011.
(60) Provisional application No. 61/522,318, filed on Aug. 11, 2011.
(60) Provisional application No. 61/577,771, filed on Dec. 20, 2011.

(51) Int. Cl. G01N 33/574; A61K 31/706; A61P 35/00; G01N 33/569; A61P 35/02; A61P 31/12; A61P 33/00; G01N 21/64; A61K 31/7068; A61P 31/00

(52) U.S. Cl. 514/49; 514/43; 435/7.21; 435/7.23

(57) ABSTRACT

This invention relates generally to methods, assays and systems for detecting hENT1 expression levels in hematological disorders, to methods, assays and systems for detecting hENT1 expression levels in hematological disorders using flow cytometry, and to methods, assays and systems for detecting hENT1 expression levels in myelodysplastic syndrome (MDS) and acute myeloid leukemia (AML) using flow cytometry. The invention also relates to diagnostic and therapeutic uses for the detection of hENT1 expression levels in a subject.

### RELATED APPLICATIONS

This application claims the benefit of U.S. Provisional Application No. 61/475,953, filed Apr. 15, 2011; U.S. Provisional Application No. 61/522,318, filed Aug. 11, 2011; and U.S. Provisional Application No. 61/577,771, filed Dec. 20, 2011. The contents of each of these applications are hereby incorporated by reference in their entirety.

### INCORPORATION-BY-REFERENCE OF SEQUENCE LISTING

The contents of the text file named “37269506001US.txt,” which was created on Apr. 12, 2012 and is 44.3 KB in size, are hereby incorporated by reference in their entirety.

### FIELD OF THE INVENTION

This invention relates generally to methods, assays and systems for detecting hENT1 expression levels in hematological disorders, to methods, assays and systems for detecting hENT1 expression levels in hematological disorders using flow cytometry, and to methods, assays and systems for detecting hENT1 expression levels in myelodysplastic syndrome (MDS) and acute myeloid leukemia (AML) using flow cytometry. The invention also relates to diagnostic and therapeutic uses for the detection of hENT1 expression levels in a subject.

### BACKGROUND OF THE INVENTION

Human equilibrative nucleoside transporter 1 (hENT1) is a protein that is encoded by the SLC29A1 gene. This gene is a member of the equilibrative nucleoside transporter family. The gene encodes a transmembrane glycoprotein that localizes to the plasma and mitochondrial membranes and mediates the cellular uptake of nucleosides from the surrounding medium. The protein is categorized as an equilibrative (as opposed to concentrative) transporter that is sensitive to inhibition by nitrobenzylthioinosine (NBMPR). Nucleoside transporters are required for nucleotide synthesis in cells that lack de novo nucleoside synthesis pathways, and are also necessary for the uptake of cytotoxic nucleosides and nucleoside analogue drugs used for cancer and viral chemotherapies.

Accordingly, there exists a need for methods that selectively detect and quantify hENT1 expression level in tumor cells.

### SUMMARY OF THE INVENTION

The present invention provides methods and compositions for measuring the levels of nucleoside transporters in a sample or a subject and correlating this level to a predicted efficacy of a given anti-cancer drug regime. The methods of the present invention allows for the treatment of cancer with a rationally selected and designed drug regime. In some aspects of the invention, the level of hENT in cancer cells is determined, and individuals with low levels of hENT1 are treated with an anti-cancer drug such as a nucleoside analog drug and/or a drug that is derived from a nucleoside analog. For example, the anti-cancer drug is selected from pyrimidine derivatives including, for example, cytarabine, gemcitabine, azacytidine, and derivatives thereof, and purine derivatives including, for example, fludarabine, cladribine, clofarabine and derivatives thereof. In some embodiments, the anti-cancer drug is a lipophilic gemcitabine analog such as gemcitabine-5′-elaidate, a lipophilic cytarabine analog such as cytarabine-5′-elaidate or a lipophilic azacytidine analog such as azacytidine-5′ elaidate. Other suitable pyrimidine analogs include, by way of non-limiting example, azacytidine-5′-petroselinate, decitabine-5′-elaidate, decitabine-5′-petroselinate and ribavirin-5′-elaidate. Other suitable purine analogs include, by way of non-limiting example, clofarabine-5′-elaidate, clofarabine-5′-petroselinate, fludarbine-5′-elaidate, fludarabine-5′-petroselinate, cladribine-5′-elaidate and cladribine-5′-petroselinate.

In some embodiments, the analogs are useful as anti-infectious disease compounds such as, for example, anti-viral compounds and/or anti-parasitic compounds including anti-malarial compounds. (See e.g., Quashie et al., “Uptake of purines in Plasmodium falciparum-infected human erythrocytes is mostly mediated by the human equilibrative nucleoside transporter and the human facilitative nucleobase transporter,” Malaria Journal, vol. 9: 36 (2010)). In these embodiments, the invention provides methods for treating, delaying the progression of, preventing a relapse of, alleviating a symptom of, or otherwise ameliorating an infectious disease in a subject, e.g., a human subject, by detecting hENT1 expression level in the subject and comparing the hENT1 expression level in the subject with a control level of hENT1 expression level; and administering an effective dose of an anti-infectious disease drug to ameliorate the infectious disease in the subject exhibiting a decreased level of hENT1 expression.

In some embodiments, the control level of hENT1 expression is derived from a ratiometric index comparing hENT1 expression levels in one or more of the leukemic blast cells, monocytes, granulocytes and eosinophils to hENT1 expression levels in normal autologous lymphocytes.

The invention provides methods for treating, delaying the progression of, preventing a relapse of, alleviating a symptom of, or otherwise ameliorating a cancer in a subject, e.g., a human subject, by detecting hENT1 expression level in the subject and comparing the hENT1 expression level in the subject with a control level of hENT1 expression level, and administering an effective dose of an anti-cancer drug to ameliorate the cancer in the subject exhibiting a decreased level of hENT1 expression. The hENT1 expression level in the subject is determined, for example, by detecting the level of hENT1 expressed by a population of cells in the subject. For example, in some embodiments, the hENT1 expression level in the subject is determined by detecting and quantifying the level of hENT1 expressed by tumor cells. In some embodiments, the hENT1 expression level in the subject is determined by detecting and quantifying the level of hENT1 expressed by infected cells, such as, for example, virally infected cells or cells infected with a parasite.

The invention provides methods for treating cancer in an individual by determining the level of nucleoside transporter in a sample derived from an individual in need of the treatment of a cancer, and transmitting data pertaining to the nucleoside transporter level to a physician who provides an instruction regarding administering a therapeutically effective amount of an anti-cancer drug, for example, a chemotherapeutic nucleoside analog, to the individual based on the nucleoside transporter level.

In some embodiments, the amount of the anti-cancer drug is determined based upon the level of hENT1 expression. In some embodiments, the amount of the anti-cancer drug is determined based upon the level of hENT1 expression as compared to a control level of hENT1 expression. In some embodiments, the control level of hENT1 expression is derived from a ratiometric index comparing hENT1 expression levels in one or more of the leukemic blast cells, monocytes, granulocytes and eosinophils to hENT1 expression levels in normal autologous lymphocytes.

In some embodiments, a particular anti-cancer drug is administered based upon the level of hENT1 expression. In some embodiments, the amount of the anti-cancer drug is determined based upon the level of hENT1 expression as compared to a control level of hENT1 expression. In some embodiments, the control level of hENT1 expression is derived from a ratiometric index comparing hENT1 expression levels in one or more of the leukemic blast cells, monocytes, granulocytes and eosinophils to hENT1 expression levels in normal autologous lymphocytes.

In some embodiments, the cancer is a hematological cancer. For example, the cancer is myelodysplastic syndrome (MDS), acute myeloid leukemia (AML), acute lymphoblastic leukemia (ALL) or chronic myelomonocytic leukemia (CMML).

In some embodiments, the level of hENT1 expression is detected in a sample from a subject identified as having or as being at risk for having a hematological disorder. Suitable samples include tissues, cells and biological fluids isolated from a subject, as well as tissues, cells and fluids present within a subject. Included within the usage of the term “biological sample” herein is blood and a fraction or component of blood including blood serum, blood plasma, or lymph.

In some embodiments, the sample is bone marrow aspirate. In some embodiments, the sample is peripheral blood. In some embodiments, the sample is cerebrospinal fluid or a component thereof. In some embodiments, the sample is pleural effusion. In some embodiments, the sample is ascites.

In some embodiments, the sample is a fresh sample. For example, the sample is less than 48 hours old, e.g., less than 24 hours old.

In some embodiments, the sample is a frozen sample. In some embodiments, the frozen sample is, e.g., Ficoll density gradient separated cells that are cryopreserved by slow freezing (e.g., slow freezing) in dimethyl sulfoxide (DMSO) or other suitable solvent.

In some embodiments, the level of hENT1 expression is detected using flow cytometry.

In some embodiments, the level of hENT1 expression is detected using an antibody that specifically binds to hENT1 or an antigen-binding fragment thereof. In some embodiments, the anti-hENT1 antibody is a monoclonal antibody. In some embodiments, the anti-hENT1 antibody is a monoclonal antibody that includes a variable heavy chain sequence such the VH3-12 variable heavy chain, the VH5-9 variable heavy chain, the VH5-12 variable heavy chain, the VH5-13 variable heavy chain, or the consensus variable heavy chain provided herein and referred to as the consensus variable heavy chain region sequence 1 (consensus VH sequence 1). In some embodiments, the anti-hENT1 antibody is a monoclonal antibody that includes the VH1-1 variable heavy chain, the VH1-4 variable heavy chain, the VH1-6 variable heavy chain, the VH4-2 variable heavy chain, the VH4-3 variable heavy chain, the VH4-4 variable heavy chain or the consensus variable heavy chain provided herein and referred to as the consensus variable heavy chain region sequence 2 (consensus VH sequence 2). In some embodiments, the anti-hENT1 antibody is a monoclonal antibody that includes a light chain variable sequence such as, for example, the VL2 variable light chain, the VL10 variable light chain, the VL11 variable light chain, the VL20 variable light chain, the VL21 light chain, or the consensus variable light chain provided herein and referred to as the consensus variable light chain region sequence 1 (consensus VL sequence 1). In some embodiments, the anti-hENT1 antibody is a monoclonal antibody that includes the VL2-2 variable light chain, the VL2-3 variable light chain, the VL2-7 variable light chain, the VL2-10 variable light chain, the VL2-12 variable light chain, the VL2-16 variable light chain or the consensus variable light chain provided herein and referred to as the consensus variable light chain region sequence 2 (consensus VL sequence 2). These antibodies are respectively referred to herein as “hENT1 antibodies” or “anti-hENT1 antibodies”. hENT1 antibodies include fully human monoclonal antibodies, as well as humanized monoclonal antibodies and chimeric antibodies. hENT1 antibodies can include constant heavy or light chains from other species, such as, for example, rabbit, for improved stability and/or detection. These antibodies show specificity for hENT1.

In some embodiments, the hENT1 antibody includes a heavy chain variable region having the amino acid sequence of SEQ ID NOs: 2, 4, 6, 8, 9, 28, 30, 32, 34, 36, 38 or 39. In some embodiments, the hENT1 antibody includes a light chain variable region having the amino acid sequence of SEQ ID NOs: 14, 16, 18, 20, 22, 23, 43, 45, 47 or 49. In some embodiments, the hENT1 antibody includes a heavy chain variable region having an amino acid sequence at least 90%, 91%, 92%, 93%, 94%, 95%, 96%, 97% 98%, 99% or more identical the amino acid sequence of SEQ ID NOs: 2, 4, 6, 8, 9, 28, 30, 32, 34, 36, 38 or 39. In some embodiments, the hENT1 antibody includes a light chain variable region having an amino acid sequence at least 90%, 91%, 92%, 93%, 94%, 95%, 96%, 97% 98%, 99% or more identical the amino acid sequence of SEQ ID NOs: 14, 16, 18, 20, 22, 23, 43, 45, 47 or 49.

In some embodiments, the hENT1 antibody includes a variable heavy chain complementarity determining region 1 (VH CDR1) sequence comprising the amino acid sequence GYTFTDYE (SEQ ID NO: 10), a variable heavy chain complementarity determining region 2 (VH CDR2) sequence comprising the amino acid sequence IDPETGAI (SEQ ID NO: 11) or the amino acid sequence IDPETGKT (SEQ ID NO: 40), and a variable heavy chain complementarity determining region 3 (VH CDR3) sequence comprising the amino acid sequence TREFTY (SEQ ID NO: 12) or the amino acid sequence TRELTY (SEQ ID NO: 41).

In some embodiments, the hENT1 antibody includes a variable heavy chain complementarity determining region 1 (VH CDR1) that includes an amino acid sequence at least 87% or more identical to the amino acid sequence GYTFTDYE (SEQ ID NO: 10); a variable heavy chain complementarity determining region 2 (VH CDR2) that includes an amino acid sequence at least 87% or more identical to the amino acid sequence IDPETGAI (SEQ ID NO: 11) or the amino acid sequence IDPETGKT (SEQ ID NO: 40); and a variable heavy chain complementarity determining region 3 (VH CDR3) that includes an amino acid sequence at least 83% or more identical to the amino acid sequence TREFTY (SEQ ID NO: 12) or the amino acid sequence TRELTY (SEQ ID NO: 41).

In some embodiments, the hENT1 antibody includes a variable light chain complementarity determining region 1 (VL CDR1) sequence comprising an amino acid sequence at least 90% or more identical to the amino acid sequence QSLLFSNGKTY (SEQ ID NO: 24), a variable light chain complementarity determining region 2 (VL CDR2) sequence comprising an amino acid sequence at least 66% or more identical to the amino acid sequence LVS (SEQ ID NO: 25), and a variable light chain complementarity determining region 3 (VL CDR3) sequence comprising an amino acid sequence at least 88% or more identical to the amino acid sequence VQGTHFPWT (SEQ ID NO: 26).

In some embodiments, the hENT1 antibody includes a variable light chain complementarity determining region 1 (VL CDR1) sequence comprising the amino acid sequence QSLLFSNGKTY (SEQ ID NO: 24), a variable light chain complementarity determining region 2 (VL CDR2) sequence comprising the amino acid sequence LVS (SEQ ID NO: 25), and a variable light chain complementarity determining region 3 (VL CDR3) sequence comprising the amino acid sequence VQGTHFPWT (SEQ ID NO: 26).

In some embodiments, the level of hENT1 expression is compared to a control level of hENT1 expression.

In some embodiments, the control level is an “internal control,” such as, for example, using normal cells in a sample that express consistently high/intermediate or low levels of hENT1. In some embodiments, the control level of hENT1 expression is derived from a ratiometric index comparing hENT1 expression levels in one or more of the leukemic blast cells, monocytes, granulocytes and eosinophils to hENT1 expression levels in normal autologous lymphocytes. Alternatively or in addition, the control level is an “external control,” such as, for example a cell line that has been engineered to express hENT1 at a given level, e.g., the cell line CCRF-CEM (expresses approximately 100,000-300,000 hENT1 transporters per cell) and/or CEM/ara-C lacking hENT1.

In some embodiments, the control level has previously been determined from a source other than the subject. In some embodiments, the control level of hENT1 is contemporaneously determined from a source other than the subject. Suitable sources for these embodiments include any of those described herein.

In some embodiments, the control level is determined by obtaining a second non-cancerous sample from the subject. In some embodiments, the control level is determined by obtaining a non-cancerous sample from a different subject. Suitable samples include tissues, cells and biological fluids isolated from a subject, as well as tissues, cells and fluids present within a subject. Included within the usage of the term “biological sample” herein is blood and a fraction or component of blood including blood serum, blood plasma, or lymph.

In some embodiments, the control level of hENT1 expression is derived from a ratiometric index comparing hENT1 expression levels in one or more of the leukemic blast cells, monocytes, granulocytes and eosinophils to hENT1 expression levels in normal autologous lymphocytes.

In some embodiments, the control level is determined using the level of hENT1 expression in multiple control sources. Suitable control sources include any of those described herein.

In some embodiments, the control level is determined by obtaining a statistical distribution of hENT1 levels.

In some embodiments, the control level is determined from cultured cells engineered to express hENT1. In some embodiments, the control level is determined from cells engineered to not express hENT1. Suitable cell types include cells and cell lines recognized in the art as suitable for cell culture and/or any cells described herein.

In some embodiments, the control level is a clinically accepted reference level.

In some embodiments, the level of hENT1 expression in the subject is classified as high, medium or low according to an H-Score.

In some embodiments, the level of hENT1 expression in the subject is classified as a low sample when the H-Score is less than or equal to the overall median H-Score.

In some embodiments, effective dose of the anti-cancer drug is administered as a single dose. In some embodiments, the effective dose of the anti-cancer drug is administered as multiple doses.

In some embodiments, the subject is non-responsive, less responsive or has stopped responding to treatment with a chemotherapeutic agent. For example, in some embodiments, the chemotherapeutic agent is gemcitabine, cytarabine and/or azacytidine. Other suitable chemotherapeutic agents include those recognized in the art, any of the chemotherapeutic and anti-neoplastic agents described herein and/or any of the anti-infectious disease agents described herein.

In some embodiments, the anti-cancer drug and/or pharmaceutical compositions thereof is administered in combination with any of a variety of known therapeutics, including for example, chemotherapeutic and other anti-neoplastic agents, anti-inflammatory compounds and/or immunosuppressive compounds. In some embodiments, the anti-cancer drug and/or pharmaceutical compositions thereof is useful in conjunction with any of a variety of known treatments including, by way of non-limiting example, surgical treatments and methods, radiation therapy, chemotherapy and/or hormone or other endocrine-related treatment.

In some embodiments, the anti-cancer drug is administered in combination with one or more chemotherapeutic and/or cytotoxic agents. Suitable agents and/or cytotoxic agents include those recognized in the art and/or any of the chemotherapeutic agents, anti-neoplastic agents and/or cytotoxic agents described herein.

These “co-therapies” can be administered sequentially or concurrently. The anti-cancer drug and/or pharmaceutical compositions thereof and the additional agent(s) can be administered to a subject, preferably a human subject, in the same pharmaceutical composition. Alternatively, the anti-cancer drug and/or pharmaceutical compositions thereof and the second agent(s) can be administered concurrently, separately or sequentially to a subject in separate pharmaceutical compositions. The anti-cancer drug and/or pharmaceutical compositions thereof and the second therapy may be administered to a subject by the same or different routes of administration.

In some embodiments, the co-therapies of the invention comprise an effective amount of the anti-cancer drug and/or pharmaceutical compositions thereof and an effective amount of at least one other therapy (e.g., prophylactic or therapeutic agent) which has a different mechanism of action than the gemcitabine, cytarabine and/or azacytidine analogs described herein, e.g., gemcitabine-5′-elaidate, cytarabine-5′-elaidate and/or azacytidine-5′ elaidate. In some embodiments, the co-therapies of the present invention improve the prophylactic or therapeutic effect of the anti-cancer drug and of the second therapy by functioning together to have an additive or synergistic effect. In some embodiments, the co-therapies of the present invention reduce the side effects associated with the second therapy (e.g., prophylactic or therapeutic agents).

In some embodiments, the anti-cancer drug is administered in combination with the additional agent(s). The term “in combination” in this context means that the anti-cancer drug and the additional agent(s) are given substantially contemporaneously, either simultaneously or sequentially. If given sequentially, at the onset of administration of the second compound, the first of the two compounds is preferably still detectable at effective concentrations at the site of treatment. The anti-cancer drug can be administered first and the additional agent(s) can be administered second, or alternatively, the additional agent(s) can be administered first and the anti-cancer drug can be administered second.

In some embodiments, combination therapy can include one or more anti-cancer drugs such as, azacytidine-5′ elaidate, cytarabine-5′ elaidate and/or gemcitabine-5′-elaidate, coformulated with one or more additional agents.

The anti-cancer drugs and additional agent(s) can be administered by the same or by different routes of administration.

In some embodiments, the anti-cancer drug, combination therapy and/or pharmaceutical compositions thereof is administered orally in a dosage form, for example, a tablet, pill, capsule (hard or soft), caplet, powder, granule, suspension, solution, gel, cachet, troche, lozenge, syrup, elixir, emulsion, oil-in-water emulsion, water-in-oil emulsion, and/or a draught.

Administration of the anti-cancer drug, combination therapies, and/or pharmaceutical compositions thereof to a patient suffering from a cell proliferation disease or disorder is considered successful if any of a variety of laboratory or clinical results is achieved. For example, administration is considered successful if one or more of the symptoms associated with the cell proliferation disease or disorder is alleviated, reduced, inhibited or does not progress to a further, i.e., worse, state. Administration is considered successful if the cell proliferation disorder, e.g., cancer or other neoplastic condition, enters remission and/or does not progress to a further, i.e., worse, state.

The invention also provides methods of directing treatment of a disease by delivering a sample suspected of having a low level of functional hENT1 to a diagnostic lab for determination of hENT1 levels; providing a control sample with a known level of hENT; providing an antibody or other means of detecting hENT1; subjecting the sample and control sample to binding by the antibody or other anti-hENT1 agent, and detecting a relative amount of antibody or anti-hENT1 agent binding, wherein a sample with a low amount of hENT1 binding is used to provide a conclusion that a patient should receive a particular anti-cancer drug such as azacytidine-5′ elaidate, cytarabine-5′ elaidate or gemcitabine-5′-elaidate.

The invention also provides methods of directing treatment of a disease further comprises reviewing or analyzing data relating to the presence of hENT1 in a sample; and providing a conclusion to an individual, a health care provider or a health care manager, the conclusion being based on the review or analysis of data. In one aspect of the invention a conclusion is the transmission of the data over a network.

The invention provides methods of detecting a level of hENT1 expression in a subject by contacting a sample from the subject with one or more antibodies that bind a cell surface marker or cell marker under conditions sufficient to allow binding between the antibody and the cell surface markers, wherein the sample contains at least leukemic blast cells, lymphocytes and at least one or more additional types of normal, non-leukemic blood cells selected from monocytes, granulocytes and eosinophils; permeabilizing the cells from the sample; contacting the permeabilized cells from the sample with an antibody that binds hENT1 under conditions sufficient to allow binding between the antibody and hENT1, wherein the anti-hENT1 antibody is detectably labeled with a fluorophore, such as, for example, FITC and/or PE; detecting by flow cytometry the fluorescence level of: (i) the one or more additional types of normal, non-leukemic blood cells from the sample; (ii) the lymphocytes from the sample; and (iii) the leukemic blast cells from the sample; determining a control ratio level of hENT1 expression in the sample by comparing the fluorescence level of each additional type of normal, non-leukemic blood cell in (i) with the fluorescence level of the lymphocytes in (ii); determining the ratio level of hENT1 expression in the leukemic blast cells from the sample by comparing the fluoresce level of the leukemic blast cells in (iii) with the fluorescence level of the lymphocytes in (ii); and comparing the ratio level of hENT1 expression in the leukemic blast cells with the control ratio level to determine the level of hENT1 expression in the subject.

In some embodiments, hENT1 expression level is quantified by a ratiometric index comparing hENT1 levels in leukemic blast cells, monocytes (mono), granulocytes (gran) and eosinophils (eos) to the hENT1 levels of normal autologous lymphocytes (lymph). Ratiometric methods are based on the use of a ratio between two fluorescence intensities and are not affected by variations in conditions that may affect the assay (such as, by way of non-limiting example, instrument to instrument differences, levels of hENT1 in normal cells, non-specific binding). Therefore, using ratios avoids many of the problems related to absolute fluorescence values.

In some embodiments, the ratios in the assays provided herein are calculated as follows:

| | Divided by Lymph | |
|---|---|---|
| Cell Population | Population | =Cell hENT1 Index |
| Mono Median X | Lymph Median X | =Mono hENT1 Index |
| Gran Median X | Lymph Median X | =Gran hENT1 Index |
| Eos Median X | Lymph Median X | =Eos hENT1 Index |
| Blast (ROI) Median X | Lymph Median X | =Blast hENT1 Index |

In some embodiments, the subject is suffering from acute myeloid leukemia (AML). In some embodiments, the hematological disorder is myelodysplastic syndrome (MDS), acute lymphoblastic leukemia (ALL) or chronic myelomonocytic leukemia (CMML).

In some embodiments, the cell surface marker or cell marker is selected from CD45, CD163, CD33, CD34, CD38, CD123, CD117, CD13, CD64, HLA-DR, myeloperoxidase (MPO) and combinations thereof. In some embodiments, the one or more antibodies that bind a cell surface marker or cell marker are detectably labeled, for example, with a fluorophore. Suitable fluorophores include, by way of non-limiting examples, FITC, PE, PerCP-Cy5.5 and/or PE-Cy7.

In some embodiments, the anti-hENT1 antibody includes a variable heavy chain complementarity determining region 1 (VH CDR1) sequence comprising the amino acid sequence GYTFTDYE (SEQ ID NO: 10); a variable heavy chain complementarity determining region 2 (VH CDR2) sequence comprising the amino acid sequence IDPETGAI (SEQ ID NO: 11) or the amino acid sequence IDPETGKT (SEQ ID NO: 40); a variable heavy chain complementarity determining region 3 (VH CDR3) sequence comprising the amino acid sequence TREFTY (SEQ ID NO: 12) or the amino acid sequence TRELTY (SEQ ID NO: 41); a variable light chain complementarity determining region 1 (VL CDR1) sequence comprising the amino acid sequence QSLLFSNGKTY (SEQ ID NO: 24); a variable light chain complementarity determining region 2 (VL CDR2) sequence comprising the amino acid sequence LVS (SEQ ID NO: 25); and a variable light chain complementarity determining region 3 (VL CDR3) sequence comprising the amino acid sequence VQGTHFPWT (SEQ ID NO: 26).

In some embodiments, the anti-hENT1 antibody includes a heavy chain variable sequence comprising an amino acid sequence selected from SEQ ID NO: 2, 4, 6, 8, 9, 28, 30, 32, 34, 36, 38 and 39, and a light chain variable sequence comprising the amino acid sequence selected from SEQ ID NO: 14, 16, 18, 20, 22, 23, 43, 45, 47 and 49.

In some embodiments, the methods of the invention include an immunogenic hENT1 peptide. For example, in some embodiments, the flow cytometry assays provided herein include an immunogenic hENT1 peptide. As used herein, the terms “immunogenic hENT1 peptide” and “antigenic peptide,” used interchangeably herein, refer to a hENT1 protein, polypeptide and/or peptide that retains the ability to provoke or otherwise stimulate an immune response in a patient. In some embodiments, the immunogenic hENT1 peptide is a fragment of the full-length hENT1 protein. In some embodiments, the immunogenic hENT1 peptide is a fragment of the human full-length hENT1 protein. For example, the immunogenic hENT1 peptide is a fragment of a full-length hENT1 protein having a sequence, for example, as shown in GenBank Accession Nos. AAC51103.1; NP_{—}001071645.1; NP_{—}001071644.1; NP_{—}0010171643.1; NP_{—}001071642.1; NP_{—}004946.1; NP_{—}001523.2; AAM11785.1; AAF02777.1). For example, the immunogenic hENT1 peptide is a fragment of the human full-length hENT1 protein comprising at least a portion of the predicted intracellular loop between transmembrane segments 6 and 7 of the hENT1 protein. (See e.g., Zhang et al. “The role of nucleoside transporters in cancer chemotherapy with nucleoside drugs,” Cancer Metastasis Rev. 26 (2007): 85-110. For example, the immunogenic hENT1 peptide is a fragment of the human full-length hENT1 protein comprising at least the sequence SKGEEPRAGKEESGVSVS, which correspond to amino acids 254-271 of the predicted intracellular loop between transmembrane segments 6 and 7 of the hENT1 protein shown in FIG. 2. In some embodiments, the immunogenic hENT1 peptide fragment comprises at least 5, 10, 15, 20, 25, 30, 35, 40, 45, 50, 55 or 60 amino acids of the of the predicted intracellular loop between transmembrane segments 6 and 7 of the hENT1 protein shown in FIG. 2.

In some embodiments, the immunogenic hENT1 peptide is a fragment of a full-length hENT1 human protein. For example, the immunogenic hENT1 peptide fragment is 2%, 3%, 4%, 5%, 6%, 7%, 8%, 9%, 10%, 15%, 20%, 25%, 30%, 35%, 40%, 45%, 50% or more than 50% identical to a full-length human hENT1 protein, such as, for example, those shown in GenBank Accession Nos. AAC51103.1; NP_{—}001071645.1; NP_{—}001071644.1; NP_{—}0010171643.1; NP_{—}001071642.1; NP_{—}004946.1; NP_{—}001523.2; AAM11785.1; AAF02777.1).

Use of an immunogenic hENT1 peptide in these flow cytometry assays allows for the measurement of specific binding levels of the hENT1 antibody to the intracellular antigen. The flow cytometry assays that include an immunogenic hENT1 peptide are advantageous over other assays that do not include this peptide. For example, these immunogenic hENT1 peptides can be used for quantification of hENT1 levels in a patient or sample. These immunogenic hENT1 peptides can also be used for normalization of the quantification of hENT1 levels in a patient or sample. In addition, these immunogenic hENT1 peptides address a common problem in flow cytometry assays that do not include such peptides—the ability to detect or otherwise identify whether the antibody binding is specific or non-specific. The inclusion of an immunogenic hENT1 peptide demonstrates that the detected levels of hENT1 are due to specific binding between the antibodies and hENT1, rather than non-specific binding.

In one embodiment, the flow cytometry assay is run in at least two parallel tubes, wherein one tube does not include an immunogenic hENT1 peptide, and another tube does contain an immunogenic hENT1 peptide at a specific concentration. The relative or percentage inhibition by the peptide is then used as a means of quantification. Calibration beads are spiked in the sample as internal reference. The different normal cell populations can be used as controls. The data presented in the Examples below indicates that the signal differs between the different cell types.

The invention also provides methods of detecting the level of a target protein, polypeptide and/or peptide in a sample using flow cytometry. In some embodiments, these methods of the invention also include an immunogenic version of the target peptide. As used herein, the terms “immunogenic target peptide,” immunogenic peptide” and/or “antigenic peptide” refer to a target protein, polypeptide and/or peptide that retains the ability to provoke or otherwise stimulate an immune response in a patient. In some embodiments, the immunogenic peptide is a fragment of the full-length target protein. In some embodiments, the immunogenic peptide is a fragment of the human full-length target protein. For example, the immunogenic peptide is a fragment of the human full-length target protein comprising at least a portion of an extracellular domain of the target protein. In some embodiments, the immunogenic peptide fragment comprises at least 5, 10, 15, 20, 25, 30, 35, 40, 45, 50, 55, 60, 65, 70, 75, 80, 85, 90, 95, 100 or more than 100 amino acids of the full length target protein.

In some embodiments, the immunogenic peptide is a fragment of a full-length human target protein. For example, the immunogenic peptide fragment is 2%, 3%, 4%, 5%, 6%, 7%, 8%, 9%, 10%, 15%, 20%, 25%, 30%, 35%, 40%, 45%, 50% or more than 50% identical to a full-length human target protein.

Use of an immunogenic peptide in these flow cytometry assays allows for the measurement of specific binding levels of the anti-target antibody to the target antigen. The flow cytometry assays that include an immunogenic peptide are advantageous over other assays that do not include such a peptide. For example, these immunogenic peptides can be used for quantification of the level of target in a patient or sample. These immunogenic peptides can also be used for normalization of the quantification of target levels in a patient or sample. In addition, these immunogenic to detect or otherwise identify whether the antibody binding is specific or non-specific. The inclusion of an immunogenic peptide demonstrates that the detected levels of target are due to specific binding between the antibodies and the target, rather than non-specific binding.

In one embodiment, the flow cytometry assay is run in at least two parallel tubes, wherein one tube does not include an immunogenic target peptide, and another tube does contain an immunogenic target peptide at a specific concentration. The relative or percentage inhibition by the peptide is then used as a means of quantification. Calibration beads are spiked in the sample as internal reference. The different normal cell populations can be used as controls.

### BRIEF DESCRIPTION OF THE DRAWINGS

FIG. 1 is a schematic representation of an embodiment of a flow cytometry method for detecting hENT1 expression levels in a sample.

FIG. 2 is a schematic representation of the topology of hENT1 from Zhang et al., “The role of nucleoside transporters in cancer chemotherapy with nucleoside drugs,” Cancer Metastasis Rev., vol. 26: 85-110 (2007) with additional illustrations depicting the principles of flow assay methods of the invention.

FIG. 3 is an illustration depicting the reduction in signal in a normal sample (referred to as Normal #1) seen in the presence of an immunogenic hENT1 peptide (bottom two rows of panels) as compared to the level of signal detected in the absence of the immunogenic hENT1 peptide (top two rows of panels).

FIG. 4 is a graph depicting the percent inhibition exhibited by normal cell populations of monocytes, granulocytes, lymphocytes and eosinophils.

FIG. 5 is a graph depicting the difference between the ratio of the cell population median to beads in the presence or absence of an immunogenic hENT1 peptide.

FIGS. 6A-6D are a series of graphs depicting the inhibition of anti-hENT1 antibody signal by a BSA-conjugated hENT1 peptide in (A) PMN cells, (B) monocytes, (C) lymphocytes and (D) eosinophils.

FIG. 7 is a graph depicting the variability in the ratiometric index of hENT1 expression for AML blasts in two different flow cytometer instruments.

### DETAILED DESCRIPTION

hENT1 is a trans/membrane protein abundant in most healthy cells as well as in many tumor cells. As hENT1 is ubiquitously expressed, it is important that the methods provided herein selectively detect and quantify hENT1 in tumor cells, including subsets of cells such as blast cells.

The invention provides a method for treating a patient with a hematological cancer or other disorder. The method comprises predicting an individual's response to a cancer therapy, including a response by human cancer patients. Methods for matching a particular chemotherapeutic agent to particular individual based on predicted efficacy, and methods for directing treatment and informing patients and physicians are also provided.

Nucleoside analogue drugs depend on nucleoside transporters to enter the cells where they exert their effect—these molecules do not cross the plasma membrane by diffusion, and efficient cellular uptake requires the presence of these specialized plasma membrane nucleoside transporter proteins.

Cytarabine and gemcitabine (Gemzar, Eli Lilly, Indianapolis, Ind.) are two anticancer drugs that depend on human Equilibrative Nucleoside Transporter 1 (hENT1) for their effect. A considerable proportion of cancer cells have low expression of hENT1. Low expression and/or activity of hENT1 has been found in patients having cancers and other neoplastic disorders such as, for example, acute myeloid leukemia (AML) or pancreatic cancer. Low clinical effect of treatment has been correlated with reduced or no presence of hENT1 in the cancer cells. (See e.g., Galmarini, et al., “Potential mechanisms of resistance to cytarabine in AML patients,” Leuk Res 26 (2002) 621-629; Farrell, et al., “Human Equilibrative Nucleoside Transporter 1 Levels Predict Response to Gemcitabine in Patients With Pancreatic Cancer,” Gastroenterology 136 (2009) 187-195; and Giovannetti, et al., “Transcription analysis of human equilibrative nucleoside transporter-1 predicts survival in pancreas cancer patients treated with gemcitabine,” Cancer Res 66 (2006) 3928-3935). Studies have shown a direct correlation between the lack of hENT1 expression on a pancreatic cancer patient's tumor cells and that patient's poor response to nucleoside analogue and other chemotherapeutic drugs such as gemcitabine. (See Farrell, et al., “Human Equilibrative Nucleoside Transporter 1 Levels Predict Response to Gemcitabine in Patients With Pancreatic Cancer,” Gastroenterology 136 (2009) 187-195). hENT1 is also important for the oral uptake of ribavirin and can cause resistance to ribavirin hepatitis C treatment. (See Ibarra et al., “Reduced ribavirin antiviral efficacy via nucleoside transporter-mediated drug resistance,” J. Virol., vol. 83(9): 4583-47 (2009)).

In the methods provided herein, a population of patients with low or otherwise reduced hENT1 expression and/or activity is identified for additional or otherwise altered treatment regimens. For example, this identified patient population is administered a treatment that is designed to allow uptake in hENT1 deficient cells, such as for example, lipid-conjugated gemcitabine derivatives such as gemcitabine-5′-elaidic acid ester, lipid-conjugated cytarabine derivatives such as cytarabine-5′-elaidic acid ester, or lipid-conjugated azacytidine derivatives such as 5-azacytidine-5′-elaidic acid ester. (See Brueckner, et al., “Delivery of 5-azacytidine to human cancer cells by elaidic acid esterification increases therapeutic drug efficacy,” Mol Cancer Ther., vol. 9(5): 1256-1264 (2010)). Studies have shown that these drugs, in contrast to established nucleoside drugs such as gemcitabine, cytarabine and azacytidine, are able to enter cancer cells and retain their activity in cancer cells independent of the hENT1 expression level in the cancer cell. (See e.g. Breistol, et al., “Antitumor activity of P-4055 (elaidic acid-cytarabine) compared to cytarabine in metastatic and s.c. human tumor xenograft models,” Cancer Res 59 (1999) 2944-2949; and Galmarini et al., “CP-4055 and CP-4126 are active in ara-C and gemcitabine-resistant lymphoma cell lines,” Br J Haematol 144 (2009) 273-275).

The uptake of gemcitabine-5′-elaidic acid ester and cytarabine-5′-elaidic acid ester in hENT1 deficient cells has been confirmed also in vitro with confirmed high formation of the active triphosphate metabolite of cytarabine-5′-elaidic acid ester and gemcitabine-5′-elaidic acid ester in deficient cancer cells. Once inside the cell the lipid tail of the lipid-conjugated drug such as gemcitabine-5′-elaidic acid ester or cytarabine-5′-elaidic acid ester is cleaved off, and the parent drug is released. With the lack of hENT1 transporter (due to the low expression and/or activity of hENT1 in the identified patient population), the drug is trapped inside the cell, and high concentrations of active metabolites have been measured. (See Adema et al., “Metabolism and accumulation of the lipophilic deoxynucleoside analogs elacytarabine and CP-4126, Invest. New Drugs, 2011 Oct. 15. [Epub ahead of print]).

These observations indicate that lipid-conjugated gemcitabine derivatives such as gemcitabine-5′-elaidic acid ester, lipid-conjugated azacytidine derivatives such as azacytidine-5′-elaidate, and/or lipid-conjugated cytarabine derivatives such as cytarabine-5′-elaidic acid ester are useful in treating tumors that are resistant or otherwise less responsive to cytarabine, azacytidine and gemcitabine due to the lack of hENT1 or low hENT1 expression and/or activity.

In some embodiments, the patients are currently receiving treatment regimens that include administration of one or more nucleoside analogue drugs and/or drugs derived from nucleoside analogues, such as, pyrimidine derivatives including, for example, cytarabine, gemcitabine, azacytidine, and derivatives thereof, and purine derivatives including, for example, fludarabine, cladribine, clofarabine and derivatives thereof. In some embodiments, the patients are currently receiving treatment regimens that include administration of one or more nucleoside analogue drugs and/or drugs derived from nucleoside analogues, such as pyrimidine derivatives including, for example, cytarabine, gemcitabine, azacytidine, and derivatives thereof, and purine derivatives including, for example, fludarabine, cladribine, clofarabine and derivatives thereof, and these patients have stopped responding to treatment or are otherwise less responsive to the nucleoside analogue drug.

In some embodiments, the patients have previously received treatment regimens that included administration of one or more nucleoside analogue drugs and/or drugs derived from nucleoside analogues, such as pyrimidine derivatives including, for example, cytarabine, gemcitabine, azacytidine, and derivatives thereof and purine derivatives including, for example, fludarabine, cladribine, clofarabine and derivatives thereof. In some embodiments, the patients have previously received treatment regimens that included administration of one or more nucleoside analogue drugs and/or drugs derived from nucleoside analogues, such as pyrimidine derivatives including, for example, cytarabine, gemcitabine, azacytidine, and derivatives thereof and purine derivatives including, for example, fludarabine, cladribine, clofarabine and derivatives thereof, and these patients stopped responding to treatment or were otherwise less responsive to the nucleoside analogue drug.

In some embodiments, the patients are de novo patients and the methods provided herein provide and/or assist in the initial diagnosis. For example, in patients at risk or otherwise suspected of having AML, the level of hENT1 expression is used to predict response to cytarabine and to determine whether the patient should receive a cytarabine analog such as cytarabine-5′ elaidate. The detected level of hENT1 expression is used to predict the patient's response and/or outcome to treatment with an anti-drug compound or an analog thereof. Thus, in this embodiment, the methods provided herein are useful in assisting with the initial diagnosis and initial therapeutic treatment decision.

### Methods

In order to treat a cancer patient, the present invention teaches the following general aspects. First, a hematological cancer patient is identified, or a patient suspected of having or being at risk for a hematological cancer is identified. Individuals are identified using any known diagnostic technique. The identification can be performed by a physician. The identification of the individual can be by communication with the physician, the individual, a health care company, an insurer, or from a computer database which stores data related to the individual. In some embodiments, the identification of the individual is concurrent with the testing of the samples. In some embodiments, the individual is identified and then further testing is performed. As used herein, the term “individual” is synonymous with “a patient” or “a subject”. In some embodiments, the individual is suspected of having cancer. In some embodiments, the individual has been diagnosed with cancer. In some embodiments, the individual has been proven to have cancer. In some embodiments, the individual is a human, however in some embodiments the individual is a non-human mammal. In some embodiments, the non-human mammal is a domesticated animal with cancer. In some embodiments, the individual is in the midst of an ongoing therapeutic regime. In some embodiments, the individual has not yet received treatment. In some embodiments, the individual is subjected to a diagnostic test in the midst of an ongoing therapeutic regime so as to identify levels of transporters such as hENT1 or hCNTI in cancerous cells or tissue.

Second, a sample containing cancer cells is obtained from this individual and analyzed to determine the level of expression of one or more nucleoside transporters. In some embodiments, the nucleoside transporter level is determined in a sample of a bodily fluid. In some embodiments, a bodily fluid sample is taken from an individual and nucleoside transporter levels are obtained from the bodily fluid. In some embodiments, the levels of nucleoside transporter are obtained from a subset of cells obtained in a bodily fluid sample. Bodily fluids include but are not limited to blood, lymph, saliva, semen, CSF, breast milk, peritoneal fluid, and pleural effusion.

Third, this information is then used to determine which of the available anticancer drugs the patient should use. For instance, a patient that lacks or has low levels of nucleoside transporters is informed that hydrophilic nucleoside anticancer drugs are not likely to be efficacious as these drugs are not likely to enter the cancer cells. Such a patient can be given derivatives of these drugs which have been modified to enter the cancer cells independent of the transporters such as, for example, gemcitabine-5′-elaidate, cytarabine-5′-elaidate and/or azacytidine-5′ elaidate.

The methods of the invention preferably detect hENT1 expression levels using flow cytometry. Flow cytometry is the ideal platform for detection and quantification of hENT1 in cancers such as AML, MDS, ALL and CMML and in other hematological disorders, as blast cells have variable phenotypic expression and can be present as a minority cell population in patients. Flow cytometry is routinely used in the clinical diagnostic workup of these patients, and first line treatment is normally initiated as soon as the flow cytometry and immunophenotyping results are known, e.g., typically within 24-48 hours. The methods provided herein are easily integrated into current clinical practices in the treatment of AML, MDS, ALL, CMML and/or other hematological disorders. The methods provided herein are designed to be used in lieu of, in conjunction with or otherwise as a supplement to current methods of treatment for AML, MDS, ALL, CMML and other hematological disorders.

While flow cytometry has been used in previous methods of diagnosing cancer, the methods described herein provide advantageous improvements over the current uses of flow cytometry. For example, flow cytometry has been used as part of a measuring technique in detecting nucleoside transport sites in relation to ara-C activity in acute leukemias (see e.g., Wiley, J. S., Jones, S. P., Sawyer, W. H., and Paterson, A. R. (1982) Cytosine arabinoside influx and nucleoside transport sites in acute leukemia, J Clin Invest 69, 479-489). In contrast to the methods presented herein, this method described by Wiley et al. requires that, prior to analysis of the nucleoside transporter sites, the cells must be separated, for example by sedimentation gradient such as Ficoll gradient separation. Similarly, flow cytometry has been used in conjunction with SAENTA-Fluorescein to detect the number of nucleoside transporter sites in the blood of AML patients (see e.g., Gati, W. P., Paterson, A. R., Larratt, L. M., Turner, A. R., and Belch, A. R. (1997) Sensitivity of acute leukemia cells to cytarabine is a correlate of cellular es nucleoside transporter site content measured by flow cytometry with SAENTA-fluorescein, Blood 90, 346-353). This method described by Gati et al., however, also requires Ficoll gradient separation of the cells prior to analysis. Moreover, the methods described by Gati et al. require an elaborate and detailed calibration curve, as well as a larger sample size from patients to be able to evaluate the number of binding sites in a patient.

Unlike these previous methods that incorporate flow cytometry, the methods described herein provide an easy to use method that is readily integrated with standard diagnostic procedures for AML. The flow cytometry methods provided herein do not require that the cells be separated by Ficoll gradient separation (or other cell separation) prior to sample analysis. Moreover, the flow cytometry methods provided herein do not require a separate calibration curve be run when varying amounts of analytical agent are used, while the methods of Wiley et al. and Gati et al. require the use of a calibration curve for different amounts of NBMPR or SAENTA-fluorescein. In contrast to previous flow cytometry methods, the methods described herein use an anti-hENT1 antibody to provide a robust, reliable and reproducible analysis of the ratiometric index of hENT1 expression in various cell types. Accordingly, the methods described herein provide advantageous and unexpected improvements over current flow cytometry analysis methods.

In the methods provided herein, flow cytometry is used to gate or otherwise identify and separate a particular subpopulation of cells, such as, for example, leukocyte subpopulations including blast cells, from other cells within a patient sample. The methods use antibodies that are directed against specific surface markers to identify the relevant blast cells and to measure hENT1 in the sample. In some embodiments, the cells in a patient sample are permeabilized to allow access to the intracellular part of the hENT1 membrane protein to which the anti-hENT1 antibody was raised.

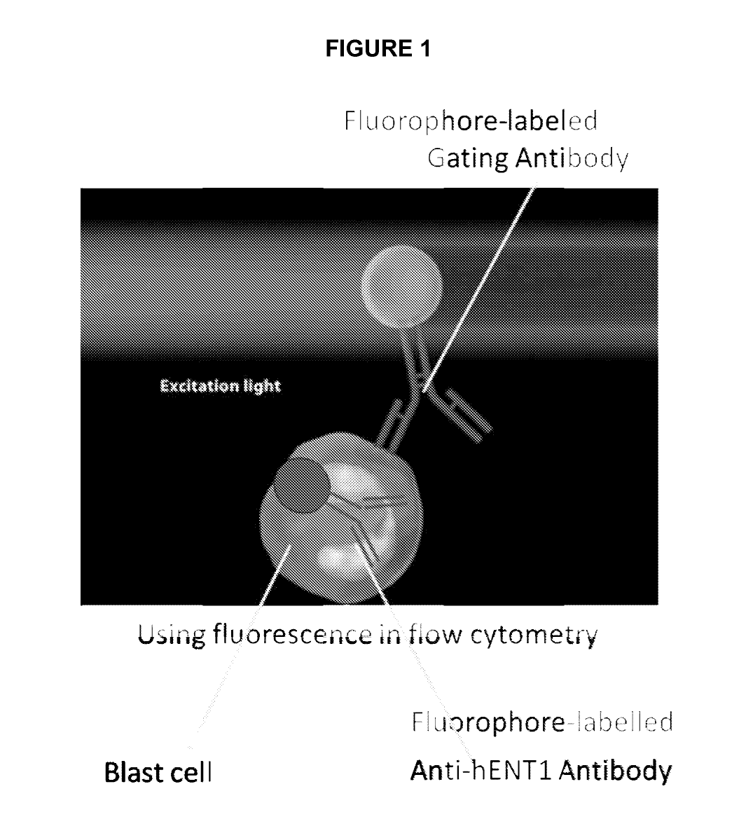

FIG. 1 provides a schematic representation of the flow cytometry assays and methods used herein to detect hENT1 expression. The assay includes the following steps:
1) Identify and gate blast cells (i.e., cancer cells) from other cells in a sample using cocktail of antibodies that detect blast cell surface markers (also referred to herein as the “gating antibody cocktail”) and are detectably labeled by a first detection means such as, a fluorophores such as R-phycoerythrin (PE) or fluorescein isothiocyanate (FITC) or other suitable dyes including, by way of non-limiting example, Alexa Fluor dyes, DyLight dyes, Cy dyes, tandem dyes including, e.g. PE-Cy5 and PE-Cy7, Pacific Blue/Orange, Lucifer yellow, Texas Red, and/or Allophycocyanin (APC);
2) Permeabilize the blast cells identified in step 1) to allow access to the inner cytoplasmic hENT1 protein; and
3) Detect and quantify hENT1 expression using an antibody that specifically binds to and recognizes hENT1, where the anti-hENT1 antibody is detected labeled with a second detection means, e.g., a fluorophores, provided that the first detection means used in step 1) is different than the second detection means used with the anti-hENT1 antibody.

The use of the different dyes with compatible spectra in the methods provided herein allows for the simultaneous detection of the different dyes as the flow cytometer has several detectors (overlapping spectrums can be compensated). Thus, the methods provided herein allow for multi-parametric analysis from one sample run. In other words, a one tube reaction is performed, thus simplifying current methods of analysis and detection.

### Gating for Cell Subpopulations

In the methods provided herein, antibodies that are directed against specific surface markers are used to identify the relevant cell subpopulation such as, for example, blast cells, from a sample or a subject. As the blast cells from different hematological disorders can be in different development stages, the methods provided herein use a variety of different antibodies against specific surface markers to identify and gate for the blast cells. Antibodies can be directed against any surface marker known in the art. For example, antibodies for use in the methods provided herein can recognize and bind to surface markers such as CD45 (a monocyte marker), CD163 (a pan leukocyte marker), CD33, CD34, CD38, CD123, CD117, CD13, CD64, HLA-DR, myeloperoxidase (MPO, a cell marker in the lysosome that is used to identify leukemic cells derived from the myeloid lineage) and any combinations thereof.

In some embodiments, the markers for use in identifying and gating or otherwise separating cells associated with AML, MDS, ALL, CMML or any other hematological disorders include one or more of the following CD1a; CD2; CD3; cCD3; CD3 (m); CD4; CD5; CD7; CD8; CD10; CD10; CD11b; CD11c; CD13; CD14; CD15; CD16; CD19; CD20; CD22 (s or c); CD23; CD24; CD25; CD30; CD33; CD34; CD35/36; CD38; CD41; CD43; CD44; CD45; CD52; CD56; CD57; CD58; CD61; CD64; CD65; CD68 (c); CD71; CD79a, including cCD79a; CD79b; CD81; CD86; CD87; CD94; CD99; CD103; CD117; CD123; CD138; cytoplasmic heavy chains; cytoplasmic light chains; DR; FMC7; granzyme B; Ig; IgM (c); δ (kappa light chain); K/L (kappa IgG light chain to lambda IgG light chain ratio); L (lambda light chain); LZ (lysozyme); MPO; MPO, alone or in combination with LF (lactoferrin); MPO/LF in combination with CD14; NK panel excluding CD19 and CD3 cells; perforin; an RBC marker such as CD238 (glycophorin A) or CD36; slg; TCR chains for T-ALL, c and/or s; TdT; uPAR(CD87)/uPACD116; or any combination thereof. (See e.g., Béné et al., “Proposals for the immunological classification of acute leukemias. European Group for the Immunological Characterization of Leukemias (EGIL),” Leukemia. vol. 9:1783-6 (1995); Lacombe, et al. “Flow cytometry CD45 gating for immunophenotyping of acute myeloid leukemia,” Leukemia. vol. 11:1878-86 (1997); Ratei et al., “Immunophenotype and clinical characteristics of CD45-negative and CD45-positive childhood acute lymphoblastic leukemia,” Ann. Hematol. vol. 77:107-114 (1998); Knapp et al., “Flow cytometric analysis of cell-surface and intracellular antigens in leukemia diagnosis, Cytometry. vol. 18:187-98 (1994); Strobl H et al. “Myeloperoxidase expression in CD34+ normal human hematopoietic cells,” Blood. vol. 1; 82:2069-78 (1993); Strobl H et al., “Identification of CD68+lin− peripheral blood cells with dendritic precursor characteristics,” J. Immunol. vol. 161:740-8 (1998); Scholz Wet al., “Initial human myeloid/dendritic cell progenitors identified by absence of myeloperoxidase protein expression,” Exp Hematol. vol. 32:270-6 (2004); Strobl H, et al., “Flow cytometric analysis of intracellular CD68 molecule expression in normal and malignant haemopoiesis,” Br J. Haematol. vol. 90:774-82 (1995); Scheinecker C, et al., “Granulomonocyte-associated lysosomal protein expression during in vitro expansion and differentiation of CD34+ hematopoietic progenitor cells,” Blood. vol. 86:4115-23 (1995); Di Noto Ret al. “All-trans retinoic acid (ATRA) and the regulation of adhesion molecules in acute myeloid leukemia,” Leuk Lymphoma. vol. 21:201-9 (1996); Paietta E. “Expression of cell-surface antigens in acute promyelocytic leukaemia,” Best Pract Res Clin Haematol. vol. 16:369-85 (2003). Braylan R C et al. “Optimal number of reagents required to evaluate hematolymphoid neoplasias: results of an international consensus meeting,” Cytometry. vol. 46:23-7 (2001); Castaneda VL, et al. “Childhood undifferentiated leukemia with early erythroid markers and c-myb duplication,” Leukemia. vol. 5:142-9 (1991); Veltroni M, Det al. “I-BFM-ALL-FCM-MRD-Study Group. Expression of CD58 in normal, regenerating and leukemic bone marrow B cells: implications for the detection of minimal residual disease in acute lymphocytic leukemia,” Haematologica. vol. 88:1245-52 (2003); Strobl H and Knapp W. “Myeloid cell-associated lysosomal proteins as flow cytometry markers for leukocyte lineage classification,” J Biol Regul Homeost Agents. vol. 18:335-9 (2004); Garand R, et al. “Minimally differentiated erythroleukaemia (AML M6 ‘variant’): a rare subset of AML distinct from AML M6. Groupe Francais d'Hematologie Cellulaire,” Br J Haematol. vol. 90:868-7 (1995); Nakahata T and Okumura N.” Cell surface antigen expression in human erythroid progenitors: erythroid and megakaryocytic markers,” Leuk Lymphoma. vol. 13:401-9 (1994); Maeda A et al. “The expression of co-stimulatory molecules and their relationship to the prognosis of human acute myeloid leukaemia: poor prognosis of B7-2-positive leukaemia,” Br J Haematol. vol. 102:1257-62 (1998); Dworzak M N, et al., “CD99 expression in T-lineage ALL: implications for flow cytometric detection of minimal residual disease,” Leukemia. vol. 18:703-8 (2004); Jacob M C, et al., “CD4+CD56+ lineage negative malignancies: a new entity developed from malignant early plasmacytoid dendritic cells,” Haematologica. vol. 88:941-55 (2003); Schott G, et al., “Immunophenotypic and clinical features of T-cell receptor (TCR) γδ+ T-lineage acute lymphoblastic leukemia (T-ALL),” Br. J. Haematol. vol. 101:753-755 (1998); Nabhan C and Rosen S T. “Conceptual aspects of combining rituximab and Campath-1H in the treatment of chronic lymphocytic leukemia,” Semin Oncol. Vol. 29(1 Suppl 2):75-80 (2002); Mulford DA and Jurcic JG. “Antibody-based treatment of acute myeloid leukaemia,” Expert Opin Biol Ther. vol. 4:95-105 (2004); Linenberger M L. “CD33-directed therapy with gemtuzumab ozogamicin in acute myeloid leukemia: progress in understanding cytotoxicity and potential mechanisms of drug resistance,” Leukemia. vol. 19:176-82 (2005); Alexander R L et al. “High affinity interleukin-3 receptor expression on blasts from patients with acute myelogenous leukemia correlates with cytotoxicity of a diphtheria toxin/IL-3 fusion protein,” Leuk Res. vol. 25:875-81 (2001); Ramage J G, et al. “The diphtheria toxin/urokinase fusion protein (DTAT) is selectively toxic to CD87 expressing leukemic cells,” Leuk Res. vol. 27:79-84 (2003); Gadhoum Z, et al. “CD44: a new means to inhibit acute myeloid leukemia cell proliferation via p27Kip1,” Blood. vol. 103:1059-68 (2004); Abi-Habib R J, et al. “A urokinase-activated recombinant diphtheria toxin targeting the granulocyte-macrophage colony-stimulating factor receptor is selectively cytotoxic to human acute myeloid leukemia blasts,” Blood. vol. 104:2143-8 (2004); Krober A, et al. “V(H) mutation status, CD38 expression level, genomic aberrations, and survival in chronic lymphocytic leukemia,” Blood vol. 100:1410-6 (2002); Laane E et al., “Flow cytometric immunophenotyping including Bcl-2 detection on fine needle aspirates in the diagnosis of reactive lymphadenopathy and non-Hodgkin's lymphoma,” Cytometry B Clin Cytom. vol. 64:34-42 (2005); Thornton P D et al., “CD38 expression as a prognostic indicator in chronic lymphocytic leukaemia,” Hematol J. vol. 5:145-51 (2004); Crespo M, et al. “ZAP-70 expression as a surrogate for immunoglobulin-variable-region mutations in chronic lymphocytic leukemia,” N Engl J Med vol. 348:1764-75 (2003); Attygalle A D, et al., “CD10 expression in extranodal dissemination of angioimmunoblastic T-cell lymphoma,” Am J Surg Pathol. vol. 28:54-61 (2004); Lee P S et al., “Immunophenotyping of angioimmunoblastic T-cell lymphomas by multiparameter flowcytometry,” Pathol Res Pract. vol. 199:539-45 (2003); Perez-Andres M, et al., “Spanish Network on multiple myeloma (G03/136); the Spanish Network of Cancer Research Centers (C03/10). Clonal plasma cells from monoclonal gammopathy of undetermined significance, multiple myeloma and plasma cell leukemia show different expression profiles of molecules involved in the interaction with the immunological bone marrow microenvironment,” Leukemia. vol. 19:449-55 (2005); Vergez et al., “High levels of CD34⁺CD38^{low/-}CD123⁺ blasts are predictive of an adverse outcome in acute myeloid leukemia: a Groupe Ouest-Est des Leucémies Aiguës et Maladies du Sang (GOELAMS) study,” Haematologica. vol. 96(12):1792-1798 (2011)).

In some embodiments, the methods provided herein are run on at least 4-color flow cytometers to be as widely applicable for clinical diagnostics, including, for example, the following cytometers: Navios and FC-500 (both from Beckman Coulter) and Canto II and FACSCalibur (both from Becton Dickenson). In some embodiments, the methods provided herein are run on 6- or more color flow cytometry may be used.

The antibodies used herein to identify and gate the blast cells using the cell surface markers or cell markers (e.g., MPO) described herein are commercially available with a range of fluorophores (e.g. FITC, PE, APC and tandem dyes such as PE-Cy5, PE-Cy7). For example, the lysosomal enzyme MPO can be identified by a fluorophore labeled anti-MPO antibody, and the anti-MPO antibody can be included either in the “gating antibody cocktail” or applied to the sample after permeabilization.

Suitable dyes and other detectable means used for gating or otherwise separating the cells include fluorophores such as R-phycoerythrin (PE) or fluorescein isothiocyanate (FITC). Other suitable dyes include, by way of non-limiting example, Alexa Fluor dyes, DyLight dyes, Cy dyes, tandem dyes including, e.g. PE-Cy5 and PE-Cy7, Pacific Blue/Orange, Lucifer yellow, Texas Red, and/or Allophycocyanin (APC).

Other suitable dyes and detectable means include any of the fluorochromes shown below in Table 1, which has been adapted from the website for The Center for Cytometry and Molecular Imaging at the Salk Institute. Table 1 depicts the characteristics (excitation wavelength (Ex), emission wavelength (Em) and molecular weight (MW)) of fluorochromes that are useful for flow cytometry or fluorescence microscopy. The fluorochromes in Table 1 are presented approximately in order of excitation wavelength.

TABLE 1
Table of Fluorochromes

| | | | |
|---|---|---|---|
| Probe | Ex (nm) | Em (nm) | MW |

Reactive and conjugated probes

| | | | |
|---|---|---|---|
| Hydroxycoumarin | 325 | 386 | 331 |
| Aminocoumarin | 350 | 445 | 330 |
| Methoxycoumarin | 360 | 410 | 317 |
| Cascade Blue | (375); 401 | 423 | 596 |
| Pacific Blue | 403 | 455 | 406 |
| Pacific Orange | 403 | 551 | |
| Lucifer yellow | 425 | 528 | |
| NBD | 466 | 539 | 294 |
| R-Phycoerythrin (PE) | 480; 565 | 578 | 240 k |
| PE-Cy5 conjugates | 480; 565; 650 | 670 | |
| PE-Cy7 conjugates | 480; 565; 743 | 767 | |
| Red 613 | 480; 565 | 613 | |
| PerCP | 490 | 675 | |
| TruRed | 490, 675 | 695 | |
| FluorX | 494 | 520 | 587 |
| Fluorescein | 495 | 519 | 389 |
| BODIPY-FL | 503 | 512 | |
| TRITC | 547 | 572 | 444 |
| X-Rhodamine | 570 | 576 | 548 |
| Lissamine | 570 | 590 | |
| Rhodamine B | | | |
| Texas Red | 589 | 615 | 625 |
| Allophycocyanin | 650 | 660 | 104 k |
| (APC) | | | |
| APC-Cy7 conjugates | 650; 755 | 767 | |

Alexa Fluor dyes [antibody conjugates] (Molecular Probes)

| | | | |
|---|---|---|---|
| Alexa Fluor 350 | 343 | 442 | 410 |
| Alexa Fluor 405 | 401 | 421 | 1028 |
| Alexa Fluor 430 | 434 | 540 | 702 |
| Alexa Fluor 488 | 499 | 519 | 643 |
| Alexa Fluor 500 | 503 | 525 | 700 |
| Alexa Fluor 514 | 517 | 542 | 714 |
| Alexa Fluor 532 | 530 | 555 | 724 |
| Alexa Fluor 546 | 561 | 572 | 1079 |
| Alexa Fluor 555 | 553 | 568 | 1250 |
| Alexa Fluor 568 | 579 | 603 | 792 |
| Alexa Fluor 594 | 591 | 618 | 820 |
| Alexa Fluor 610 | 610 | 629 | 1285 |
| Alexa Fluor 633 | 632 | 648 | 1200 |
| Alexa Fluor 647 | 652 | 668 | 1300 |
| Alexa Fluor 660 | 663 | 691 | 1100 |
| Alexa Fluor 680 | 680 | 702 | 1150 |
| Alexa Fluor 700 | 696 | 719 | 1400 |
| Alexa Fluor 750 | 752 | 776 | 1300 |
| Alexa Fluor 790 | 782 | 804 | 1750 |

Cy Dyes

| | | | |
|---|---|---|---|
| Cy2 | 489 | 506 | 714 |
| Cy3 | (512); 550 | 570; (615) | 767 |
| Cy3B | 558 | 572; (620) | 658 |
| Cy3.5 | 581 | 594; (640) | 1102 |
| Cy5 | (625); 650 | 670 | 792 |
| Cy5.5 | 675 | 694 | 1128 |
| Cy7 | 743 | 767 | 818 |

DyLight dyes (Pierce)

| | | |
|---|---|---|
| DyLight 350 | 353 | 432 |
| DyLight 405 | 400 | 420 |
| DyLight 488 | 493 | 518 |
| DyLight 549 | 562 | 576 |
| DyLight 594 | 593 | 618 |
| DyLight 633 | 638 | 658 |
| DyLight 649 | 654 | 673 |
| DyLight 680 | 692 | 712 |
| DyLight 750 | 752 | 778 |
| DyLight 800 | 777 | 794 |

### Permeabilization

In the methods provided herein, the gated blast cells are fixed and permeabilized to allow access to the intracellular loop of hENT1 to which the anti-hENT1 antibody binds. Suitable permeabilization agents include commercially available permeabilization agents such as IntraCell™ from Trillium Diagnostics, which is a permeabilization agent based on the detergent Triton X100. Other suitable permeabilization agents include, e.g. Fix+Perm™ (Becton Dickenson), Caltag™ (Beckman coulter), Saponin or a suitable methanol-based method.

### Anti-hENT1 Antibodies

Once the cells are permeabilized to allow access to the intracellular loop of hENT1, the cells are contacted with an anti-hENT1 antibody. Preferably, the anti-hENT1 antibody is a monoclonal antibody.

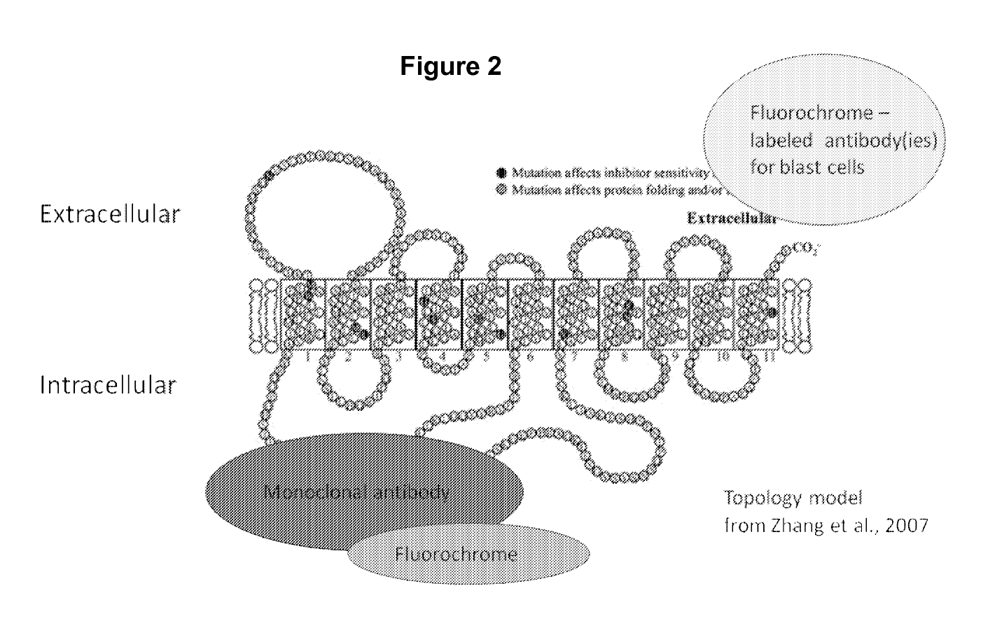

For example, the anti-hENT1 antibody is an antibody produced by the immunization of mice with a synthetic peptide (SKGEEPRAGKEESGVSVS, conjugated to KLH) that corresponded to amino acids 254-271 of the predicted intracellular loop between transmembrane segments 6 and 7 of hENT1. (See Jennings, et al, “Distinct regional distribution of human equilibrative nucleoside transporter proteins 1 and 2 (hENT1 and hENT2) in the central nervous system,” Neuropharmacology, vol. 40(5):722-31 (2001). The topology of hENT1 is shown in FIG. 2.

Exemplary anti-hENT1 antibodies include a variable heavy chain sequence selected from the following VH chains described herein: the VH3-12 variable heavy chain, the VH5-9 variable heavy chain, the VH5-12 variable heavy chain, the VH5-13 variable heavy chain, or the consensus variable heavy chain provided herein and referred to as the consensus variable heavy chain region sequence 1 (consensus VH sequence 1). Exemplary anti-hENT1 antibodies also include antibodies that include a variable heavy chain sequence selected from the following VH chains described herein: the VH1-1 variable heavy chain, the VH1-4 variable heavy chain, the VH1-6 variable heavy chain, the VH4-2 variable heavy chain, the VH4-3 variable heavy chain, the VH4-4 variable heavy chain or the consensus variable heavy chain provided herein and referred to as the consensus variable heavy chain region sequence 2 (consensus VH sequence 2). The variable domain of each heavy chain sequence is shown in bold in the sequences below. The complementarity determining regions (CDRs) are shown in boxes in the sequences below. The CDRs were identified using IMGT algorithms. (Lefranc, et al., Dev. Comp. Immunol., 27, 55-77 (2003); Brochet et al., “IMGTN-QUEST: the highly customized and integrated system for IG and TR standardized V-J and V-D-J sequence analysis,” Nucl. Acids Res., vol. 36: W503-508 (2008)).

The VH3-12 heavy chain variable region (SEQ ID NO: 2) is encoded by the nucleic acid sequence shown in SEQ ID NO: 1. The variable domain is shown in bold in the amino acid sequence shown in SEQ ID NO: 2. The CDR regions are boxed in the amino acid sequence shown in SEQ ID NO: 2.

>VH3-12 nucleic acid sequence (SEQ ID NO: 1)
ATGGAATGCACCTGGGTTTTTCTCTTCCTCCTGTCAGTAATTGCAGGTGTCCAATCCCAGGTTC
ATCTGCAGCAGTCTGGGGCTGAGCTGGTGAGGCCTGGGGCTTCAGTGACGCCGCCCTGCAAGGC
TTCGGGCTACACATTTACTGACTATGAAATGCACTGGGTGAAACAGACACCTGTGCATGGCCTG
GAATGGATTGGCGCTATTGATCCTGAAACTGGTGCTATTGTCTACAATCAGAAGTTCAAGGGCA
AGGCCACACTGACTGCAGACAAATCCTCCAACACAGCCCACATGGAGCTCCGCAGCCTGACATC
TGAGGACTCTGCCGTCTATTACTGTACAAGAGAGTTTACTTACTGGGGCCAAGGGACTCTGGTC
ACTGTCTCTGCAGCCAAAACGACACCCCCATCCGTCTTCCCCCTGGCACAAGCCAAATTCTGCA
GATATCCATCACACTGGCGGCCGCTCGAGCATCTATTCTTTGGGAA
>VH3-12 amino acid sequence (SEQ ID NO: 2)

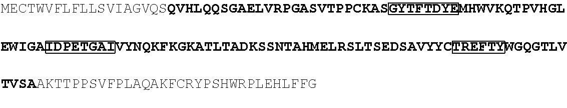

The VH5-9 heavy chain variable region (SEQ ID NO: 4) is encoded by the nucleic acid sequence shown in SEQ ID NO: 3. The variable domain is shown in bold in the amino acid sequence shown in SEQ ID NO: 4. The CDR regions are boxed in the amino acid sequence shown in SEQ ID NO: 4.

>VH5-9 nucleic acid sequence
(SEQ ID NO: 3)
ATGGAATGCACCTGGGTTATTCTCTTCCTCCTGTCAGTAATTGCAGGTGTCCAATCCCAGGTTC
ATCTGCAGCAGTCTGGGGCTGAGCTGGTGAGGCCTGGGGCTTCAGTGACGCTGCCCTGCAAGGC
TTCGGGCTACACATTTACTGACTATGAAATGCACTGGGTGAAACAGACACCTGTGCATGGCCTG
GAATGGATCGGCGCTATTGATCCTGAAACTGGTGCTATTGTCTACAATCAGAAGCTCAAGGGCA
AGGCCACACTGGCTGCAGACAAATCCTCCAACACAGCCTACATGGAGCTCCGCAGCCTGACATC
TGAGGACTCTGCCGTCTATTACTGTACAAGAGAGTTTACTTACTGGGGCCAAGGGACTCTGGTC
ACTGTCTCTGCAGCCAAAACGACACCCCCACCCGTTTATCCACTGGCCCCCTGGAAGCTTGGG
>VH5-9 amino acid sequence
(SEQ ID NO: 4)

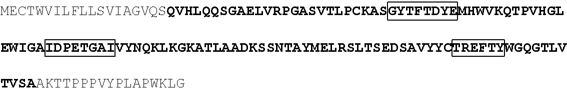

The VH5-12 heavy chain variable region (SEQ ID NO: 6) is encoded by the nucleic acid sequence shown in SEQ ID NO: 5. The variable domain is shown in bold in the amino acid sequence shown in SEQ ID NO: 6. The CDR regions are boxed in the amino acid sequence shown in SEQ ID NO: 6.

>VH5-12 nucleic acid sequence
(SEQ ID NO: 5)
ATGAAATGGACCTGGGTTTTCCTCTTCCTCCTGTCAGTAATTGCAGGTGTCCAATCCCAGGTTC
ATCTGCAGCAGTCTGGGGCTGAGCTGGTGAGGCCTGGGGCTTCAGTGACGCTGCCCTGCAAGGC
TTCGGGCTACACATTTACTGACTATGAAATGCACTGGGTGAAACAGACACCTGTGCATGGCCTG
GAATGGATTGGCGCTATTGATCCTGAAACTGGTGCTATTGTCTACAATCAGAAGTTCAAGGGCA
AGGCCACACTGACTGCAGACAAATCCTCCAACACAGCCTACATGGAGCTCCGCAGCCTGACATC
TGAGGACTCTGCCGTCTATTACTGTACAAGAGAGTTTACTTACTGGGGCCAAGGGACTCTGGTC
ACTGTCTCTGCAGCCAAAACGACACCCCCATCCGTTTATCCACTGGCCCCTGGAAGCTTGGG
>VH5-12 amino acid sequence
(SEQ ID NO: 6)

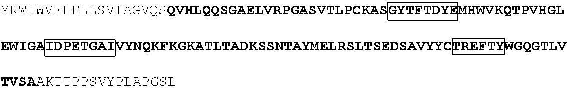

The VH5-13 heavy chain variable region (SEQ ID NO: 8) is encoded by the nucleic acid sequence shown in SEQ ID NO: 7. The variable domain is shown in bold in the amino acid sequence shown in SEQ ID NO: 8. The CDR regions are boxed in the amino acid sequence shown in SEQ ID NO: 8.

>VH5-13 nucleic acid sequence
(SEQ ID NO: 7)
ATGGAATGCAGCAGGGTTATTCTCTTCCTCCTGTCAGTAATTGCAGGTGTCCAATCCCAGGTTC
ATCTGCAGCAGTCTGGGGCTGAGCTGGTGAGGCCTGGGGCTTCAGTGACGCTGCCCTGCAAGGC
TTCGGGCTACACATTTACTGACTATGAAATGCACTGGGTGAAACAGACACCTGTGCATGGCCTG
GAATGGATTGGCGCTATTGATCCTGAAACTGGTGCTATTGTCTACAATCAGAAGTTCAAGGGCA
AGGCCACACTGACTGCAGACAAATCCTCCAACACAGCCTACATGGAGCTCCGCAGCCTGACATC
TGAGGACTCTGCCGTCTATTACTGTACAAGAGAGTTTACTTACTGGGGCCAAGGGACTCTGGTC
ACTGTCTCTGCAGCCAAAACGACACCCCCACCCGTCTATCCCCTGGCCCCTGGAAGCTTGGG
>VH5-13 amino acid sequence
(SEQ ID NO: 8)

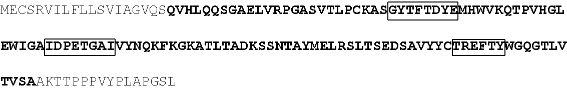

The amino acid sequence of the consensus heavy chain variable region sequence 1 is shown in SEQ ID NO: 9. The variable domain is shown in bold in the amino acid sequence shown in SEQ ID NO: 9. The CDR regions are boxed in the amino acid sequence shown in SEQ ID NO: 9.

>Consensus VH amino acid sequence 1
(SEQ ID NO: 9)

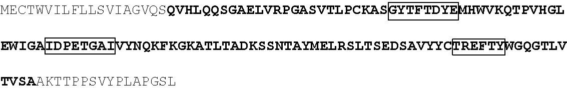

The VH1-1 heavy chain variable region (SEQ ID NO: 28) is encoded by the nucleic acid sequence shown in SEQ ID NO: 27. The variable domain is shown in bold in the amino acid sequence shown in SEQ ID NO: 28. The CDR regions are boxed in the amino acid sequence shown in SEQ ID NO: 28.

>VH1-1 nucleic acid sequence
(SEQ ID NO: 27)
ATGAAATGCAGCTGGGTTTTCCTCTTCCTCCTGTCAGTAATTGCAGGTGTCCAATCCCGGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGAAGCAGACACCTGTGCATGGCCTG
GAATGGATTGGAGCGATTGATCCTGAAACTGGTAAAACTGCCTACAATCAGAAGTTCAAGGGCA
AGACCACACTGACTGCAGACAAATCCTCCAGCACAGCCTACATGGAGTTCCGCAGCCTGACATC
TGAGGACTCTGCCGTCCATTACTGTACAAGAGAGTTGACTTACTGGGGCCAAGGGACTCTGGTC
ACTGTCTCTGCAGCCAAAACGACACCCCCATCGGTCTTCCCCCTGGCAC
>VH1-1 amino acid sequence
(SEQ ID NO: 28)

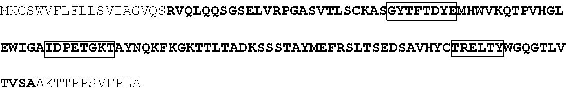

The VH1-4 heavy chain variable region (SEQ ID NO: 30) is encoded by the nucleic acid sequence shown in SEQ ID NO: 29. The variable domain is shown in bold in the amino acid sequence shown in SEQ ID NO: 30. The CDR regions are boxed in the amino acid sequence shown in SEQ ID NO: 30.

>VH1-4 nucleic acid sequence
(SEQ ID NO: 29)
ATGGAATGCACCTGGGTTTTCCTCTTCCTCCTGTCAGTAATTGCAGGTGTCCAATCCCAGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGGAGCAGACACCTGTGCATGGCCTG
GAATGGATTGGAGCGATTGATCCTGAAACTGGTAAAACTGCCTACAATCAGAAGTTCAAGGGCA
AGACCACACTGACTGCAGACAAATCCTCCAGCACAGCCTACATGGAGTTCCGCAGCCTGACATC
TGAGGACTCTGCCGTCCATTACTGTACAAGAGAGTTGACTTACTGGGGCCAAGGGACTCTGGTC
ACTGTCTCTGCAGCCAAAACGACACCCCCATCCGTCTTCCCCCTGGCAC
>VH1-4 amino acid sequence
(SEQ ID NO: 30)

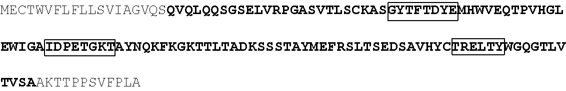

The VH1-6 heavy chain variable region (SEQ ID NO: 32) is encoded by the nucleic acid sequence shown in SEQ ID NO: 31. The variable domain is shown in bold in the amino acid sequence shown in SEQ ID NO: 32. The CDR regions are boxed in the amino acid sequence shown in SEQ ID NO: 32.

>VH1-6 nucleic acid sequence
(SEQ ID NO: 31)
ATGAAATGCAGCTGGGTTTTCCTCTTCCTCCTGTCAGTAATTGCAGGTGTCCAATCCCAGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGAAGCAGACACCTGTGCATGGCCTG
GAATGGATTGGAGCGATTGATCCTGAAACTGGTAAAACTGCCTACAATCAGAAGTTCAAGGGCA
AGACCACACTGACTGCAGACAAATCCCCCAGCACAGCCTACATGGAGTTCCGCAGCCTGACATC
TGAGGACTCTGCCGTCCATTACTGTACAAGAGAGTTGACTTACTGGGGCCAAGGGACTCTGGTC
ACTGTCTCTGCAGCCAAAACGACACCCCCATCGGTCTTCCCCCTGGCAC
>VH1-6 amino acid sequence
(SEQ ID NO: 32)

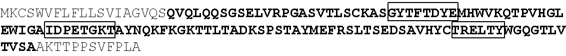

The VH4-2 heavy chain variable region (SEQ ID NO: 34) is encoded by the nucleic acid sequence shown in SEQ ID NO: 33. The variable domain is shown in bold in the amino acid sequence shown in SEQ ID NO: 34. The CDR regions are boxed in the amino acid sequence shown in SEQ ID NO: 34.

>VH4-2 nucleic acid sequence
(SEQ ID NO: 33)
ATGAAATGCAGCTGGGTTTTCCTCTTCCTCCTGTCAGTAATTGCAGGTGTCCAATCCCAGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGAAGCAGACACCTGTGCATGGCCTG
GAATGGATTGGAGCGATTGATCCTGAAACTGGTAAAACTGCCTACAATCAGAAGTTCAAGGGCA
AGACCACACTGACTGCAGACAAATCCTCCAGCACAGCCTACATGGAGTTCCGCAGCCTGACATC
TGAGGACTCTGCCGTCCATTACTGCACAAGAGAGTTGACTTACTGGGGCCAAGGGACTCTGGTC
ACTGTCTCTGCAGCCAAAACGACACCCCCACCCGTCTATCCATTGGCCCCTGGAAGCTTGGG
>VH4-2 amino acid sequence
(SEQ ID NO: 34)

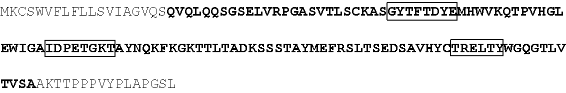

The VH4-3 heavy chain variable region (SEQ ID NO: 36) is encoded by the nucleic acid sequence shown in SEQ ID NO: 35. The variable domain is shown in bold in the amino acid sequence shown in SEQ ID NO: 36. The CDR regions are boxed in the amino acid sequence shown in SEQ ID NO: 36.

>VH4-3 nucleic acid sequence
(SEQ ID NO: 35)
ATGAAATGGACCTGGGTTTTTCTCTTCCTCCTGTCAGTAATTGCAGGTGTCCAATCCCAGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGAAGCAGACACCTGTGCATGGCCTG
GAATGGATTGGAGCGATTGATCCTGAAACTGGTAAAACTGCCTACAATCAGAAGTTCAAGGGCA
AGACCACACTGACTGCAGACAAATCCTCCAGCACAGCCTACATGGAGTTCCGCAGCCTGACATC
TGAGGACTCTGCCGTCCATTACTGTACAAGAGAGTTGACTTACTGGGGCCAAGGGACTCTGGTC
ACTGTCTCTGCAGCCAAAACGACACCCCCACCCGTCTATCCCCTGGCCCCTGGAAGCTTGGG
>VH4-3 amino acid sequence
(SEQ ID NO: 36)

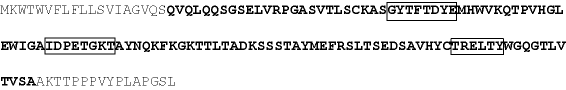

The VH4-4 heavy chain variable region (SEQ ID NO: 38) is encoded by the nucleic acid sequence shown in SEQ ID NO: 37. The variable domain is shown in bold in the amino acid sequence shown in SEQ ID NO: 38. The CDR regions are boxed in the amino acid sequence shown in SEQ ID NO: 38.

>VH4-4 nucleic acid sequence
(SEQ ID NO: 37)
ATGGAATGGAGCTGGGTTTTCCTCTTCCTCCTGTCAGTAATTGCAGGTGTCCAATCCCAGGTTC
AACTGCAGCAGTCTGGGTCTGAGCTGGTGAGGCCTGGGGCTTCAGTGACGCTGTCCTGCAAGGC
TTCGGGCTACACATTTACTGACTATGAAATGCACTGGGTGAAGCAGACACCTGTGCATGGCCTG
GAATGGATAGGAGCGATTGATCCTGAAACTGGTAAAACTGCCTACAATCAGAAGTTCAAGGGCA
AGACCACACTGACTGCAGACAAATCCTCCAGCACAGCCTACATGGAGTTCCGCAGCCTGACATC
TGAGGACTCTGCCGTCCATTACTGTACAAGAGAGTTGACTTACTGGGGCCAAGGGACTCTGGTC
ACTGTCTCTGCAGCCAAAACGACACCCCCACCCGTCTATCCATTGGCCCCCTGGAAGCTTGGG
>VH4-4 amino acid sequence
(SEQ ID NO: 38)

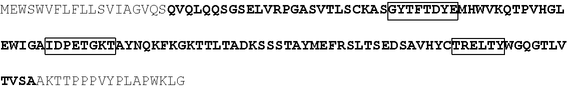

The amino acid sequence of the consensus heavy chain variable region sequence 2 is shown in SEQ ID NO: 39. The variable domain is shown in bold in the amino acid sequence shown in SEQ ID NO: 39. The CDR regions are boxed in the amino acid sequence shown in SEQ NO: 39.

>Consensus VH amino acid sequence 2 (SEQ ID NO: 39)

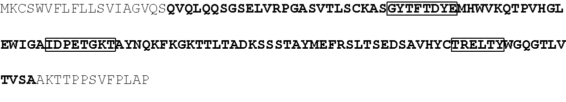

Exemplary anti-hENT1 antibodies include a variable light chain sequence selected from the following VL chains described herein: the VL2 variable light chain, the VL10 variable light chain, the VL11 variable light chain, the VL20 variable light chain, the VL21 light chain, or the consensus variable light chain provided herein and referred to as the consensus variable light chain region sequence 1 (consensus VL sequence 1). Exemplary anti-hENT1 antibodies also include antibodies that have a variable light chain sequence selected from the following VL chains described herein: the VL2-2 variable light chain, the VL2-3 variable light chain, the VL2-7 variable light chain, the VL2-10 variable light chain, the VL2-12 variable light chain, the VL2-16 variable light chain or the consensus variable light chain provided herein and referred to as the consensus variable light chain region sequence 2 (consensus VL sequence 2).

The VL2 light chain variable region (SEQ ID NO: 14) is encoded by the nucleic acid sequence shown in SEQ ID NO: 13. The variable domain is shown in bold in the amino acid sequence shown in SEQ ID NO: 14. The CDR regions are boxed in the amino acid sequence shown in SEQ ID NO: 14.

>VL2 nucleic acid sequence (SEQ ID NO: 13)
TCTCCTTTCAACAAAGCACATTTCGATTTCCAGCTTGGTGCCTCCACCGAACGTCCACGGAAAA
TGTGTACCTTGCACGCAGTAATAAACTCCCAAATCCTCAGCCTCCACTCTGCTGATTTTCAGTG
AAAAATCTGTTCCTGAACCAGTGCCAGTGAACCTGTCAGGGACTCCAGAGTTCAGTTTAGACAC
CAGATAGATTAGGCGCTTTGGAGACTGGCCTGGCCTCTGAAATAACCAATTCAAGTAGGTTTTT
CCATTACTAAATAAGAGGCTCTGACTTGACCTGCAAGAGACAGAGGCTGGTTGTCCAATGGTAA
CCGACAAAGTGAGTGGAGTTTGGGTCATCAAAACATC
>VL2 amino acid sequence (SEQ ID NO: 14)

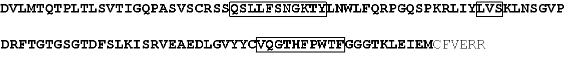

The VL10 light chain variable region (SEQ ID NO: 16) is encoded by the nucleic acid sequence shown in SEQ ID NO: 15. The CDR regions are boxed in the amino acid sequence shown in SEQ ID NO: 16.

>VL10 nucleic acid sequence (SEQ ID NO: 15)
ATTGGATATCCTCGCAGCATCTCGGCTTGATGTTTTGATGACCCAAACTCCACTCACTTTGTCG
GTTACCATTGGACAACCAGCCTCTGTCTCTTGCAGGTCAAGTCAGAGCCTCTTATTTAGTAATG
GAAAAACCTATTTGAATTGGTTATTTCAGAGGCCAGGCCAGTCTCCAAAGCGCCTAATCTATCT
GGTGTCTAAACTGAACTCTGGAGTCCCTGACAGGTTCACTGGCACTGGTTCAGGAACAGATTTT
TCACTGAAAATCAGCAGAGTGGAGGCTGAGGATTTGGGAGTTTATTACTGCGTGCAAGGTACAC
ATTTTCCGTGGACGTTCGGTGGAGGCACCAAGCTGGAAATCAAACGG
>VL10 amino acid sequence (SEQ ID NO: 16)

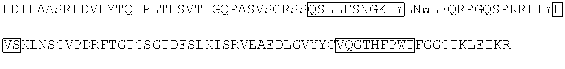

The VL11 light chain variable region (SEQ ID NO: 18) is encoded by the nucleic acid sequence shown in SEQ ID NO: 17. The CDR regions are boxed in the amino acid sequence shown in SEQ ID NO: 18.

>VL11 nucleic acid sequence (SEQ ID NO: 17)
CTTTCGCGATGATCCTTGCACGCATTTCAGGCTTGGATGTTTTGATGACCCAAACTCCACTCAC
TTTGTCGGTTACCATTGGACAACCAGCCTCTGTCTCTTGCAGGTCAAGTCAGAGCCTCTTATTT
AGTAATGGAAAAACCTATTTGAATTGGTTATTTCAGAGGCCAGGCCAGTCTCCAAAGCGCCTAA
TCTGTCTGGTGTCTAAACTGAACTCTGGAGTCCCTGACAGGTTCACTGGCACTGGTTCAGGAAC
AGATTTTTCACTGAAAATCAGCAGAGTGGAGGCTGAGGATTTGGGATTTTATTACTGCGTGCAA
GGTACACATTTTCCGTGGACGTTCGGTGGAGGCACCAAGCTGGAAATCAAACGG
>VL11 amino acid sequence (SEQ ID NO: 18)

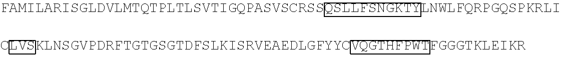

The VL20 light chain variable region (SEQ ID NO: 20) is encoded by the nucleic acid sequence shown in SEQ ID NO: 19. The variable domain is shown in bold in the amino acid sequence shown in SEQ ID NO: 20. The CDR regions are boxed in the amino acid sequence shown in SEQ ID NO: 20.

>VL20 nucleic acid sequence (SEQ ID NO: 19)
GATGTTTTGATGACCCAAACTCCACTCACTTTGTCGGTTACCATTGGACAACCAGCCTCTGTCT
CTTGCAGGTCAAGTCAGAGCCTCTTATTTAGTAATGGAAAAACCTATTTGAATTGGTTATTTCA
GAGGCCAGGCCAGTCTCCAAAGCGCCTAATCTATCTGGTGTCTAAACTGAACTCTGGAGTCTCT
GACAGGTTCACTGGCACTGGTTCAGAAACAGATTTTTCACTGAAAATCAGCAGAGTGGAGGCTG
AGGATTTGGGAGTTTATTACTGCGTGCAAGGTACATATTTTCCGTGGACGTTCGGTGGAGGCAC
CAAGCTGGAAATCAAACGGCCCTTTTTAATTCTGCAGATATCCTATCACAACGTTGCTGGCCGC
GGCCGCT
>VL20 amino acid sequence (SEQ ID NO: 20)

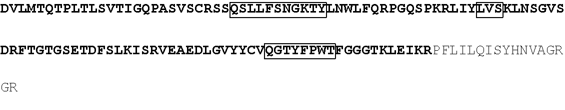

The VL21 light chain variable region (SEQ ID NO: 22) is encoded by the nucleic acid sequence shown in SEQ ID NO: 21. The variable domain is shown in bold in the amino acid sequence shown in SEQ ID NO: 22. The CDR regions are boxed in the amino acid sequence shown in SEQ ID NO: 21.

>VL21 nucleic acid sequence (SEQ ID NO: 21)
GATGTTTTGATGACCCAAACTCCACTCACTTTGTCGGTTACCATTGGACAACCAGCCTCTGTCT
CTTGCAGGTCAAGTCAGAGCCTCTTATTTAGTAATGGAAAAACCTATTTGAATTGGTTATTTCA
GAGGCCAGGCCAGTCTCCAAAGCGCCTAATCTATCTGGTGTCTAAACTGAACTCTGGAGTCCCT
GACAGGTTCACCGGCACTGGTTCAGGAACAGATTTTCCACTGAAAATCAGCAGAGTGGAGGCTG
AGGATTTGGGAGTTTATTACTGCGTGCAAGGTACACATTTTCCGTGGACGTTCGGTGCCCTTTT
TAAAGGAGGCCGTGATAAAAAAT
>VL21 amino acid sequence (SEQ ID NO: 22)

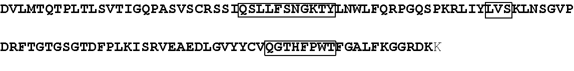

The consensus light chain variable region sequence 1 is encoded by the nucleic acid sequence shown in SEQ ID NO: 23. The CDR regions are boxed in the amino acid sequence shown in SEQ ID NO: 23.

>Consensus VL amino acid sequence 1 (SEQ ID NO: 23)

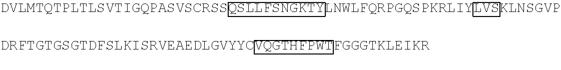

The VL2-2 light chain variable region (SEQ ID NO: 43) is encoded by the nucleic acid sequence shown in SEQ ID NO: 42. The variable domain is shown in bold in the amino acid sequence shown in SEQ ID NO: 43. The CDR regions are boxed in the amino acid sequence shown in SEQ ID NO: 43.

>VL2-2 nucleic acid sequence (SEQ ID NO: 42)
ATGAAGTTGCCTGTTAGGCTGTTGGTGCTGATGTTCTGGATTCCCGCCTCCTCCTCCGACGTGC
TGATGACCCAGACCCCCCTGACCCTGTCCGTGACCATCGGCCAGCCTGCCTCCGTGTCCTGCCG
GTCCTCCCAGTCCCTGCTGTTCTCCAACGGCAAGACCTACCTGAACTGGCTGTTCCAGCGGCCT
GGCCAGTCCCCCAAGCGGCTGATCTACCTGGTGTCCAAGCTGAACTCCGGCGTGCCCGACCGGT
TTACAGGCACCGGCTCTGGCACCGACTTCAGCCTGAAGATCAGCCGGGTGGAAGCCGAGGACCT
GGGCGTGTACTACTGCGTGCAGGGCACCCACTTCCCTTGGACCTTCGGCGGAGGCACCAAGCTG
GAAATCAAGCGGGCCGATGCCGCCCCTACCGTGTCCATCTTCCCACCCTCCAGCGAGCAGCTGA
CCTCTGGCGGCGCTTCCGTCGTGTGCTTCCTGAACAACTTCTACCCCAAAG
>VL2-2 amino acid sequence (SEQ ID NO: 43)

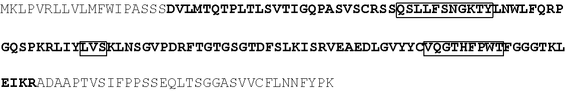

The VL2-3 light chain variable region (SEQ ID NO: 45) is encoded by the nucleic acid sequence shown in SEQ ID NO: 44. The variable domain is shown in bold in the amino acid sequence shown in SEQ ID NO: 45. The CDR regions are boxed in the amino acid sequence shown in SEQ ID NO: 45.

>VL2-3 nucleic acid sequence (SEQ ID NO: 44)
ATGAAGTTGCCTGTTAGGCTGTTGGTGCTGATGTTCTGGATTCCCGCCTCCTCCTCCGACGTGC
TGATGACCCAGACCCaCCTGACCCTGTCCGTGACCATCGGCCAGCCTGCCTCTGTGTCCTGCCG
GTCCTCCCAGTCCCTGCTGTTCTCCAACGGCAAGACCTACCTGAACTGGCTGTTCCAGCGGCCT
GGCCAGTCCCCCAAGCGGCTGATCTACCTGGTGTCCAAGCTGAACTCCGGCGTGCCCGACCGGT
TTACAGGCACCGGCTCTGGCACCGACTTCAGCCTGAAGATCAGCCGGGTGGAAGCCGAGGACCT
GGGCGTGTACTACTGCGTGCAGGGCACCCACTTCCCTTGGACCTTCGGCGGAGGCACCAAGCTG
GAAATCAAGCGGGCCGATGCCGCCCCTACCGTGTCCATCTTCCCACCCTCCAGCGAGCAGCTGA
CCTCTGGCGGCGCTTCCGTCGTGTGCTTCCTGAACAACTTCTACCCCAGAGA
>VL2-3 amino acid sequence (SEQ ID NO: 45)

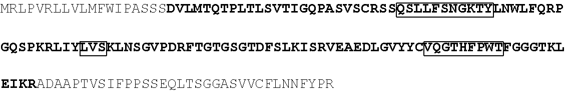

The VL2-7 light chain variable region (SEQ ID NO: 45) is encoded by the nucleic acid sequence shown in SEQ ID NO: 44. The variable domain is shown in bold in the amino acid sequence shown in SEQ ID NO: 45. The CDR regions are boxed in the amino acid sequence shown in SEQ ID NO: 45.

>VL2-7 nucleic acid sequence (SEQ ID NO: 44)
ATGAAGTTGCCTGTTAGGCTGTTGGTGCTGATGTTCTGGATTCCCGCCTCCTCCTCCGACGTGC
TGATGACCCAGACCCCCCTGACCCTGTCCGTGACCATCGGCCAGCCTGCCTCTGTGTCCTGCCG
GTCCTCCCAGTCCCTGCTGTTCTCCAACGGCAAGACCTACCTGAACTGGCTGTTCCAGCGGCCT
GGCCAGTCCCCCAAGCGGCTGATCTACCTGGTGTCCAAGCTGAACTCCGGCGTGCCCGACCGGT
TTACAGGCACCGGCTCTGGCACCGACTTCAGCCTGAAGATCAGCCGGGTGGAAGCCGAGGACCT
GGGCGTGTACTACTGCGTGCAGGGCACCCACTTCCCTTGGACCTTCGGCGGAGGCACCAAGCTG
GAAATCAAGCGGGCCGATGCCGCCCCTACCGTGTCCATCTTCCCACCCTCCAGCGAGCAGCTGA
CCTCTGGCGGCGCTTCCGTCGTGTGCTTCCTGAACAACTTCTACCCCAGAGA
>VL2-7 amino acid sequence (SEQ ID NO: 45)

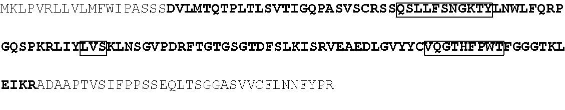

The VL2-10 light chain variable region (SEQ ID NO: 47) is encoded by the nucleic acid sequence shown in SEQ ID NO: 46. The variable domain is shown in bold in the amino acid sequence shown in SEQ ID NO: 47. The CDR regions are boxed in the amino acid sequence shown in SEQ ID NO: 47.

>VL2-10 nucleic acid sequence (SEQ ID NO: 46)
ATGAAGTTGCCTGTAGGCTGTTGGTGCTGATGTTCTGGATTCCCGCCTCCTCCTCCGACGTGCA
GATGACCCAGACCCCCCTGACCCTGTCCGTGACCATCGGCCAGCCTGCCTCTGTGTCCTGCCGG
TCCTCCCAGTCCCTGCTGTTCTCCAACGGCAAGACCTACCTGAACTGGCTGTTCCAGCGGCCTG
GCCAGTCCCCCAAGCGGCTGATCTACCTGGTGTCCAAGCTGAACTCCGGCGTGCCCGACCGGTT
TACAGGCACCGGCTCTGGCACCGACTTCAGCCTGAAGATCAGCCGGGTGGAAGCCGAGGACCTG
GGCGTGTACTACTGCGTGCAGGGCACCCACTTCCCTTGGACCTTCGGCGGAGGCACCAAGCTGG
AAATCAAGCGGGCCGATGCCGCCCCTACCGTGTCCATCTTCCCACCCTCCAGCGAGCAGCTGAC
CTCTGGCGGCGCTTCCGTCGTGTGCTTCCTGAACAACTTCTACCCCAGAGA
>VL2-10 amino acid sequence (SEQ ID NO: 47)

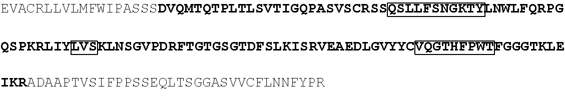

The VL2-12 light chain variable region (SEQ ID NO: 43) is encoded by the nucleic acid sequence shown in SEQ ID NO: 48. The variable domain is shown in bold in the amino acid sequence shown in SEQ ID NO: 43. The CDR regions are boxed in the amino acid sequence shown in SEQ ID NO: 43.

>V1,2-12 nucleic acid sequence (SEQ ID NO: 48)
ATGAAGTTGCCTGTTAGGCTGTTGGTGCTGATGTTCTGGATTCCCGCCTCCTCCTCCGACGTGC
TGATGACCCAGACCCCCCTGACCCTGTCCGTGACCATCGGCCAGCCTGCCTCTGTGTCCTGCCG
GTCCTCCCAGTCCCTGCTGTTCTCCAACGGCAAGACCTACCTGAACTGGCTGTTCCAGCGGCCT
GGCCAGTCCCCCAAGCGGCTGATCTACCTGGTGTCCAAGCTGAACTCCGGCGTGCCCGACCGGT
TTACAGGCACCGGCTCTGGCACCGACTTCAGCCTGAAGATCAGCCGGGTGGAAGCCGAGGACCT
GGGCGTGTACTACTGCGTGCAGGGCACCCACTTCCCTTGGACCTTCGGCGGAGGCACCAAGCTG
GAAATCAAGCGGGCCGATGCCGCCCCTACCGTGTCCATCTTCCCACCCTCCAGCGAGCAGCTGA
CCTCTGGCGGCGCTTCCGTCGTGTGCTTCCTGAACAACTTCTACCCCAAAGA
>VL2-12 amino acid sequence (SEQ ID NO: 43)

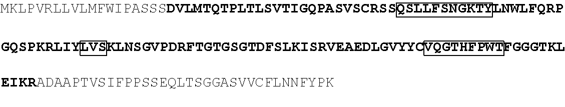

The VL2-16 light chain variable region (SEQ ID NO: 45) is encoded by the nucleic acid sequence shown in SEQ ID NO: 44. The variable domain is shown in bold in the amino acid sequence shown in SEQ ID NO: 45. The CDR regions are boxed in the amino acid sequence shown in SEQ ID NO: 45.

>VL2-16 nucleic acid sequence (SEQ ID NO: 44)
ATGAAGTTGCCTGTTAGGCTGTTGGTGCTGATGTTCTGGATTCCCGCCTCCTCCTCCGACGTGC
TGATGACCCAGACCCCCCTGACCCTGTCCGTGACCATCGGCCAGCCTGCCTCTGTGTCCTGCCG
GTCCTCCCAGTCCCTGCTGTTCTCCAACGGCAAGACCTACCTGAACTGGCTGTTCCAGCGGCCT
GGCCAGTCCCCCAAGCGGCTGATCTACCTGGTGTCCAAGCTGAACTCCGGCGTGCCCGACCGGT
TTACAGGCACCGGCTCTGGCACCGACTTCAGCCTGAAGATCAGCCGGGTGGAAGCCGAGGACCT
GGGCGTGTACTACTGCGTGCAGGGCACCCACTTCCCTTGGACCTTCGGCGGAGGCACCAAGCTG
GAAATCAAGCGGGCCGATGCCGCCCCTACCGTGTCCATCTTCCCACCCTCCAGCGAGCAGCTGA
CCTCTGGCGGCGCTTCCGTCGTGTGCTTCCTGAACAACTTCTACCCCAGAGA
>VL2-16 amino acid sequence (SEQ ID NO: 45)

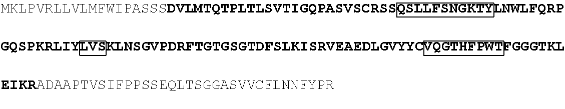

The consensus light chain variable region sequence 2 is encoded by the nucleic acid sequence shown in SEQ ID NO: 49. The variable domain is shown in bold in the amino acid sequence shown in SEQ ID NO: 49. The CDR regions are boxed in the amino acid sequence shown in SEQ ID NO: 49.

>Consensus VL amino acid sequence 2 (SEQ ID NO: 49)

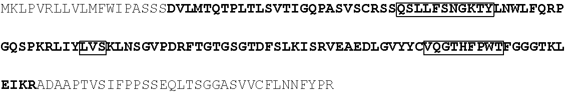

Other suitable anti-hENT1 antibodies include antibodies that bind to the same epitope as the antibodies described herein. For example, antibodies of the invention specifically bind to hENT1, wherein the antibody binds to an epitope that includes one or more amino acid residues on human hENT1 (see e.g., Accession Nos. AAC51103.1; NP_{—}001071645.1; NP_{—}001071644.1; NP_{—}0010171643.1; NP_{—}001071642.1; NP_{—}004946.1; NP_{—}001523.2; AAM11785.1; AAF02777.1).

Those skilled in the art will recognize that it is possible to determine, without undue experimentation, if a monoclonal antibody has the same specificity as a monoclonal antibody described herein by ascertaining whether the former prevents the latter from binding to hENT1. If the monoclonal antibody being tested competes with the monoclonal antibody described herein, as shown by a decrease in binding by the monoclonal antibody described herein, then the two monoclonal antibodies bind to the same, or a closely related, epitope.

An alternative method for determining whether a monoclonal antibody has the specificity of monoclonal antibody described herein is to pre-incubate the monoclonal antibody described herein with soluble hENT1 protein or a synthetic hENT1 polypeptide or peptide and then add the monoclonal antibody being tested to determine if the monoclonal antibody being tested is inhibited in its ability to bind hENT1. If the monoclonal antibody being tested is inhibited then, in all likelihood, it has the same, or functionally equivalent, epitopic specificity as the monoclonal antibody described herein.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples, including the experiments conducted and results achieved are provided for illustrative purposes only and are not to be construed as limiting upon the present invention.

### Example 1

### Detecting of hENT1 Using Flow Cytometry

In the studies presented herein, cells were stained using the following protocol. For each sample, 100 uL of whole blood was used. 10 uL of an antibody that binds a cell surface marker is added to each sample. For example, 10 uL of an anti-CD163 antibody conjugated to either FITC or PE is added to each sample, and/or 10 uL of an anti-CD45 antibody, e.g., PerCP is added to each sample. The samples were then incubated for 10 minutes at room temperature.

To each sample, 65 mL of 10% formaldehyde (3.51% final conc.) was added to fix the cells and incubated for 5 minutes at room temperature. Then, 1 mL of Intra-Cell™ was added to each sample and incubated for 30 minutes with vortexing. After 30 minutes, 2 mL of wash buffer was added to each sample, and the samples were centrifuged. The supernatant was then decanted, and 50 mL of wash buffer was added to the pellet. Then, 10 mL of a detectably labeled anti-hENT1 antibody was added to each sample and incubated for 20 minutes. For example, the anti-hENT1 antibody is labeled with a commercially available fluorophore such as PE or FITC. Studies have shown that anti-hENT1 antibodies labeled with FITC and anti-hENT1 antibodies labeled with PE are equally effective in the methods provided herein. Finally, 0.5 mL of wash buffer was added to each sample, and the samples were analyzed on the flow cytometer. (Navios, Canto II).

The total time for the staining protocol above takes no more than 60 minutes and can take as little as 30 minutes. These methods have been shown to be effective over a nearly 2 log range of hENT1 detection.

### Example 2

### Detecting of hENT1 Using Flow Cytometry in the Presence of an Immunogenic hENT1 Peptide

The following procedures were used for the hENT1 assays presented herein. First, an appropriate volume of blood was added to two tubes. Second, all samples were subject to surface antibody staining in which 10 mL CD64 PE and 10 mL CD45 PerCP were added to each tube and incubated for 10 minutes at room temperature. Third, all samples were subject to formaldehyde fixation in which 65 mL 10% formaldehyde (3.42% final concentration) was added to each tube and incubated for 5 minutes at room temperature. After 5 minutes, 1 mL of Intracell was added to each tube and incubated for 30 minutes with vortexing. After 30 minutes, 2 mL of wash buffer was added to each tube, followed by centrifugation for 2 minutes, then decanting and blotting. Each sample was washed twice in cell washer, 50 mL of wash buffer was added to each tube an vortexed. In step four, immunogenic hENT1 peptide (10 mL of 10 mg/mL 10 mg/mL BSA-peptide dilution) was added to only one tube/one sample. The other sample/other tube did not receive the peptide. Finally, each sample was stained for hENT1 by adding 60 mL appropriate dilution of hENT1 antibody to non-peptide tube and 50 mL appropriate dilution of hENT1 antibody to peptide inhibition tube. After 20 minutes of incubation, 0.5 mL wash buffer and 5 mL of Leuko64 beads were added to each sample. The samples were then analyzed on a flow cytometer.

Thus, in these studies, two tubes were processed in parallel for each donor sample: For one tube, step 4 was omitted (i.e., there was no addition of peptide) and the second tube included step 4 (i.e., peptide was added).

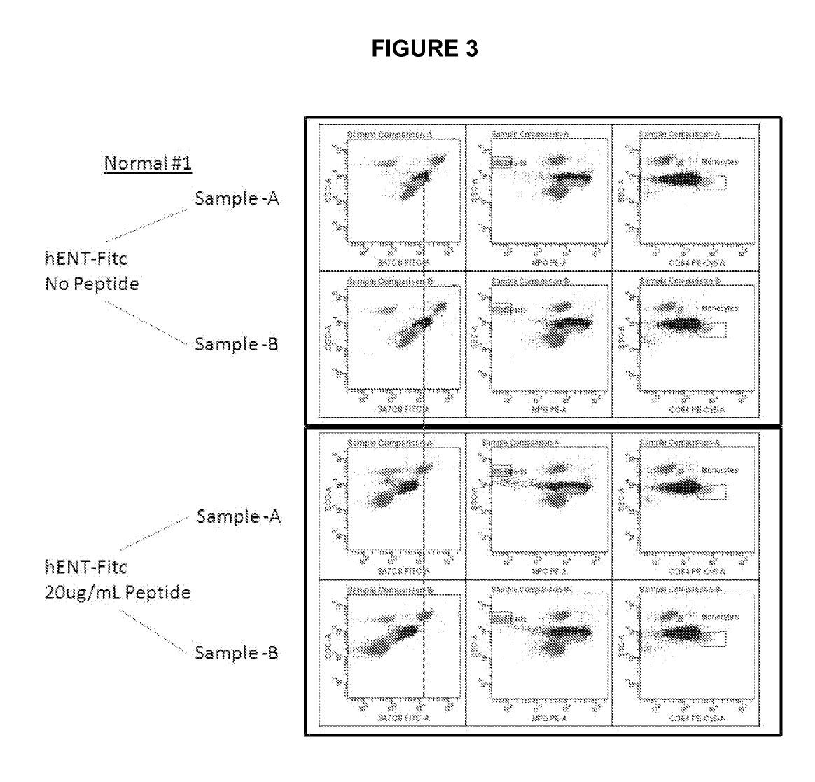

In the studies presented herein, levels of hENT1 were detected in the presence and absence of an immunogenic hENT1 peptide. As shown in FIG. 3, a reduction in signal in a normal sample (referred to as Normal #1) is seen in the presence of the immunogenic hENT1 peptide (bottom two rows of panels) as compared to the level of signal detected in the absence of the immunogenic hENT1 peptide (top two rows of panels). Other normal samples exhibited the same reduction of signal in the presence of the immunogenic hENT1 peptide.

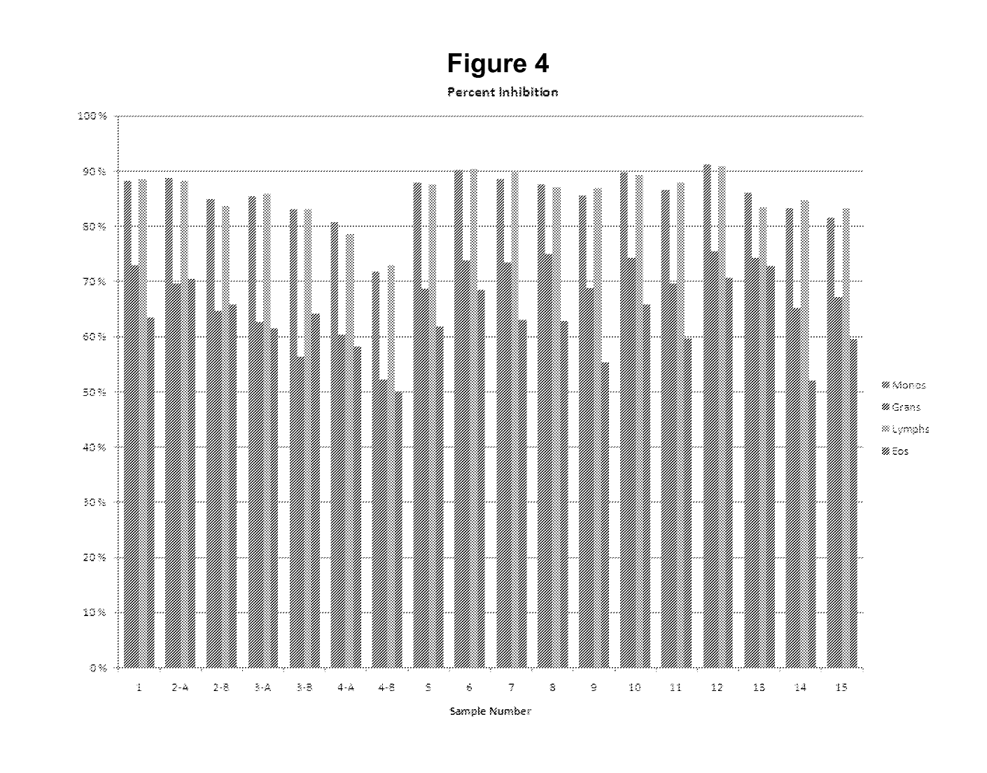

These studies indicate that different normal cell populations can be used as controls for any of the methods provided herein, as the relative or percentage inhibition by the immunogenic hENT1 peptide appears to differ between various cell types. FIG. 4 illustrates the percent inhibition exhibited by the following normal cell populations: monocytes, granulocytes, lymphocytes and eosinophils. The data shown in FIG. 5 and Table 2 below illustrate the difference between the ratio of the median signal (measured as median fluorescence intensities (MFI)) of a given cell population (monocytes, granulocytes, lymphocytes and/or eosinophils) to bead signal in the presence of the immunogenic hENT1 peptide and the ratio of the median signal (measured as MFI) of a given cell population (monocytes, granulocytes, lymphocytes and/or eosinophils) to bead signal in the absence of the immunogenic hENT1 peptide (i.e., [Signal of cell population without peptide/Signal of bead]−[Signal of cell population with peptide/Signal of bead]).

TABLE 2
Monocytes

| | | | | | | | Monos:Beads | Monos:Beads | Ratio |
|---|---|---|---|---|---|---|---|---|---|
| | | 20 ug/mL | | Percent | Monos:Lymphs | Monos:Lymphs | Ratio - No | Ratio 20 ug/mL | Difference |
| | No Peptide | Peptide | Difference | Inhibition | No Pep | With Pep | Peptide | Peptide | Monos:Beads |
| Mean | 6680.78 | 888.92 | 5791.86 | 85.65% | 2.07 | 2.08 | 8.57 | 1.13 | 6.94 |
| St. Dev. | 2014.03 | 172.23 | 1902.70 | 4.54% | 0.31 | 0.32 | 2.71 | 0.20 | 2.35 |
| CV | 30.15 | 19.38 | 32.85 | 5.30 | 14.96 | 15.34 | 31.55 | 17.93 | 33.89 |

Granulocytes

| | | | | | | | Grans:Beads | Grans: Beads | Ratio |
|---|---|---|---|---|---|---|---|---|---|
| | | 20 ug/mL | | Percent | Grans:Lymphs | Grans:Lymphs | Ratio - No | Ratio 20 ug/mL | Difference |
| | No Peptide | Peptide | Difference | Inhibition | No Pep | With Pep | Peptide | Peptide | Grans:Beads |
| Mean | 10000.92 | 3111.31 | 6889.61 | 68.06% | 3.21 | 7.36 | 12.88 | 3.98 | 7.46 |
| St. Dev. | 2603.93 | 681.33 | 2115.77 | 6.73% | 0.77 | 1.76 | 3.77 | 0.95 | 2.10 |
| CV | 26.04 | 21.90 | 30.71 | 9.89 | 23.88 | 23.96 | 29.27 | 23.95 | 28.21 |

Lymphocytes

| | | | | | Lymphs:Beads | Lymphs:Beads | |
|---|---|---|---|---|---|---|---|
| | | 20 ug/mL | | Percent | Ratio - No | Ratio 20 ug/mL | Ratio |
| | No Peptide | Peptide | Difference | Inhibition | Peptide | Peptide | Difference:Beads |
| Mean | 3250.25 | 431.17 | 2819.08 | 85.70% | 4.18 | 0.55 | 3.19 |
| St. Dev. | 924.53 | 70.12 | 864.32 | 4.46% | 1.27 | 0.09 | 1.09 |
| CV | 28.44 | 16.26 | 30.66 | 5.20 | 30.42 | 16.15 | 34.18 |

Eosinophils

| | | | | | | | Eos:Beads | Eos:Beads | |
|---|---|---|---|---|---|---|---|---|---|
| | | 20 ug/mL | | Percent | Eos:Lymphs | Eos:Lymphs | Ratio - No | Ratio 20 ug/mL | Ratio |
| | No Peptide | Peptide | Difference | Inhibition | No Pep | With Pep | Peptide | Peptide | Difference:Beads |
| Mean | 42161.06 | 15612.97 | 26382.42 | 62.56% | 13.70 | 37.04 | 54.29 | 20.03 | 30.85 |
| St. Dev. | 10785.65 | 4451.82 | 7309.71 | 6.18% | 3.97 | 10.99 | 15.90 | 6.49 | 7.63 |
| CV | 25.58 | 28.51 | 27.71 | 9.89 | 28.95 | 29.68 | 29.29 | 32.41 | 24.74 |

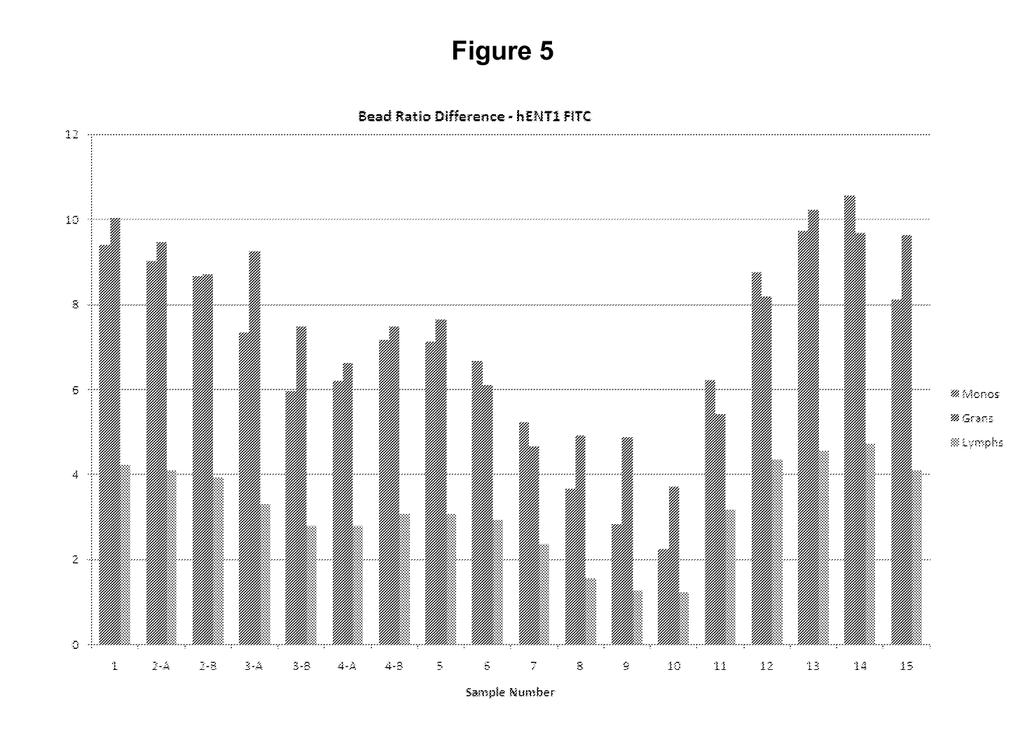

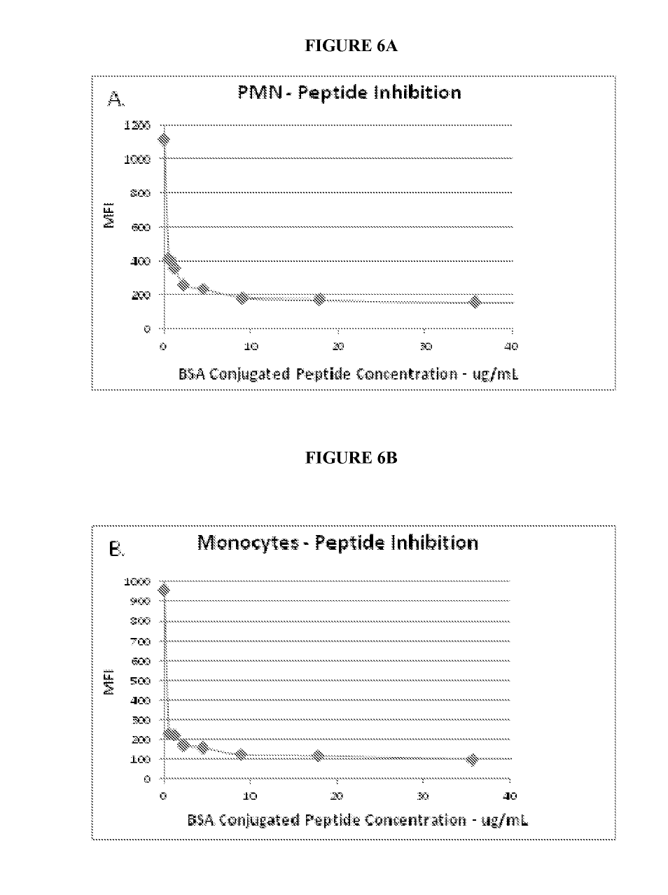

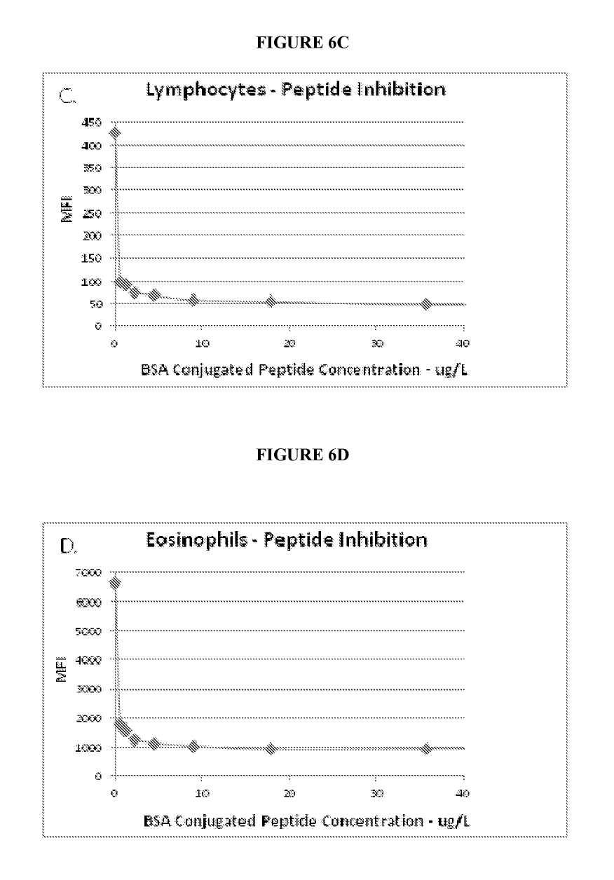

Binding of the antibody was inhibited in the presence of hENT1 peptide in a dose-dependent manner. As shown in FIGS. 6A-6D, anti-hENT1 antibody signal was inhibited by a BSA-conjugated hENT1 peptide in granulocytes (polymorphonuclear (PMN) cells, FIG. 6A), in monocytes (FIG. 6B), in lymphocytes (FIG. 6C), and in eosinophils (FIG. 6D).

### Example 3

### Detecting of hENT1 Using Flow Cytometry Using Ratiometric Methods

In this example, hENT1 expression level is quantified by a ratiometric index comparing hENT1 levels in leukemic blast cells, monocytes (mono), granulocytes (gran) and eosinophils (eos) to the hENT1 levels of normal autologous lymphocytes (lymph). As described above, ratiometric methods are based on the use of a ratio between two fluorescence intensities and are not affected by variations in conditions that may affect the assay (such as, by way of non-limiting example, instrument to instrument differences, levels of hENT1 in normal cells, non-specific binding). These methods using ratios avoid many of the problems related to absolute fluorescence values.

The ratios in the assays provided herein are calculated as follows:

| | Divided by Lymph | |
|---|---|---|
| Cell Population | Population | =Cell hENT1 Index |
| Mono Median X | Lymph Median X | =Mono hENT1 Index |
| Gran Median X | Lymph Median X | =Gran hENT1 Index |
| Eos Median X | Lymph Median X | =Eos hENT1 Index |
| Blast (ROI) Median X | Lymph Median X | =Blast hENT1 Index |

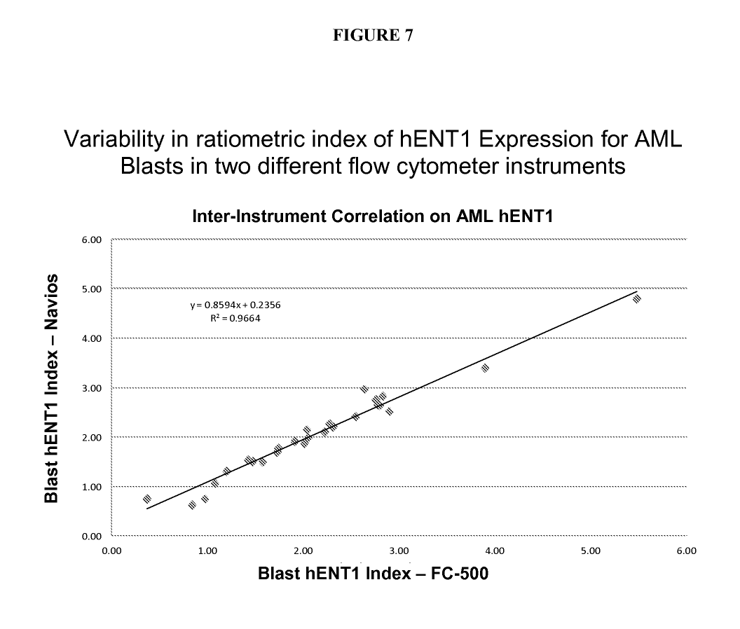

This use of ratios is a measurement principle that previously had been validated in the diagnostic flow cytometric measurement of the protein ZAP-70 expression in chronic lymphocytic leukemia cells (see e.g., Shults et. al., “A standardized ZAP-70 assay—lessons learned in the trenches.” Cytometry B Clin Cytom. July 15; 70(4) (2006):276-83; Davis and Schwartz, “ZAP-70 expression is low in normal precursor B cells or hematogones.” Cytometry B Clin Cytom. 70(4) (2006):315-9). The hENT1 assays described herein, as with many intracellular measurements by immunofluorescence methods, use a means to standardize non-specific binding as a variable between various blood and bone marrow specimens. As detailed herein, the formulation covers a range of antibody saturation conditions for a feasible cell sample specimen in the range of 1×10²-1×10¹¹ cells/ml specimen, for example, in the range of 1.5-10×10⁶ cells/ml. Within these ranges determined in the feasibility testing, the relative staining of normal cell types of lymphocytes, monocytes, neutrophilic granulocytes and eosinophils remains constant (CV<20%) for hENT1 expression. The discovery of variable hENT1 expression among normal cell types, along with the validation of a relative narrow expression variance of hENT1 on specific cell types between healthy individuals, provides the assay with a means of determining the variable expression of hENT1 in leukemic blast cells. FIG. 7 illustrates the relatively narrow expression variance of hENT1 expression index for AML Blasts using two different flow cytometer instruments. The normal cells span up to a 20 fold range of expression between the low of lymphocytes, followed by monocytes, then neutrophils to the highest level seen in eosinophils. Preliminary studies have found a nearly 10 fold range of hENT1 expression in leukemic blast cells by this assay.

Thus, the hENT1 assay is designed to report a hENT1 expression index on AML blast cells, while utilizing internal normal cell types as internal expression controls and the addition of external beads allowing for external calibration of the fluorochrome used also to label the anti-hENT1 antibody reagent.

The invention having now been described by way of written description and example, those of skill in the art will recognize that the invention can be practiced in a variety of embodiments and that the description and examples above are for purposes of illustration and not limitation of the following claims.

What is claimed is:

1. A method of ameliorating a hematological disorder in a subject, the method comprising the steps of:
a) detecting hENT1 expression level in the subject by
(i) contacting a sample from the subject with one or more agents that bind a cell surface marker or cell marker under conditions sufficient to allow binding between the antibody and the cell surface markers and selecting for the cells bound to the one or more antibodies;
(ii) permeabilizing the selected cells and contacting the permeabilized cells with an antibody that binds hENT1 under conditions sufficient to allow binding between the antibody and hENT1; and
(iii) detecting the level of permeabilized cells bound to the anti-hENT1 antibody using flow cytometry,
and comparing the hENT1 expression level in the subject with a control level of hENT1 expression level;
b) identifying a subject with a low level of hENT1 expression; and
c) administering an effective dose of an anti-cancer drug to ameliorate the hematological disorder in the subject exhibiting a decreased level of hENT1 expression, wherein the anti-cancer drug is a derivative of a nucleoside analog drug.

2. A method of ameliorating an infectious disease in a subject, the method comprising the steps of:
a) detecting hENT1 expression level in the subject by
(i) contacting a sample from the subject with one or more agents that bind a cell surface marker or cell marker under conditions sufficient to allow binding between the antibody and the cell surface markers and selecting for the cells bound to the one or more antibodies;
(ii) permeabilizing the selected cells and contacting the permeabilized cells with an antibody that binds hENT1 under conditions sufficient to allow binding between the antibody and hENT1; and
(iii) detecting the level of permeabilized cells bound to the anti-hENT1 antibody using flow cytometry,
and comparing the hENT1 expression level in the subject with a control level of hENT1 expression level;
b) identifying a subject with a low level of hENT1 expression; and
c) administering an effective dose of an anti-infectious disease drug to ameliorate the hematological disorder in the subject exhibiting a decreased level of hENT 1 expression, wherein the anti-infectious disease drug is a nucleoside analog drug or a derivative thereof.

3. The method of claim 1, wherein the anti-cancer drug or the anti-infectious disease drug is selected from gemcitabine-5′-elaidate, cytarabine-5′-elaidate, azacytidine-5′elaidate, azacytidine-5′-petroselinate, decitabine-5′-elaidate, decitabine-5′-petroselinate, ribavirin-5′-elaidate, clofarabine-5′-elaidate, clofarabine-5′-petroselinate, fludarbine-5′-elaidate, fludarabine-5′-petroselinate, cladribine-5′-elaidate and cladribine-5′-petroselinate.

4. The method of claim 1, wherein the anti-cancer drug or the anti-infectious disease drug is gemcitabine-5′-elaidate.

5. The method of claim 1, wherein the anti-cancer drug or the anti-infectious disease drug is cytarabine-5′-elaidate.

6. The method of claim 1, wherein the anti-cancer drug or the anti-infectious disease drug is azacytidine-5′elaidate.

7. The method of claim 1, wherein the hematological disorder is myelodysplastic syndrome (MDS), acute myeloid leukemia (AML), acute lymphoblastic leukemia (ALL) or chronic myelomonocytic leukemia (CMML), or wherein the infectious disease is a viral infectious disease or a parasitic infectious disease.

8. The method of claim 1, wherein the effective dose of the anti-cancer drug or the anti-infectious disease drug is administered as a single dose or as multiple doses.

9. The method of claim 1, wherein the effective dose of the anti-cancer drug or the anti-infectious disease drug is administered intravenously, subcutaneously, orally or a combination thereof.

10. The method of claim 1, wherein the subject is human.

11. The method of claim 1, wherein the subject is non-responsive, less responsive or has stopped responding to treatment with a chemotherapeutic agent or an anti-infectious disease agent.

12. The method of claim 11, wherein the chemotherapeutic agent is gemcitabine, azacytidine, cytarabine, decitabine or ribavirin.

13. The method of claim 1, wherein the gemcitabine-5′-elaidate, cytarabine-5′-elaidate or azacytidine-5′ elaidate is administered in combination with one or more additional chemotherapeutic, cytotoxic or anti-infectious disease agents.

14. The method of claim 1, wherein the control level of hENT1 expression is derived from a ratiometric index comparing hENT1 expression levels in one or more of the leukemic blast cells, monocytes, granulocytes and eosinophils to hENT1 expression levels in normal autologous lymphocytes.

15. The method of claim 1, wherein the one or more agents that bind a cell surface marker or cell marker is an antibody.

16. The method of claim 15, wherein the antibody binds to a cell surface marker or cell marker selected from CD45, CD163, CD33, CD34, CD38, CD123, CD117, CD13, CD64, HLA-DR, myeloperoxidase (MPO) and combinations thereof.

17. The method of claim 15, wherein the one or more antibodies that bind a cell surface marker or cell marker is detectably labeled.

18. The method of claim 17, wherein the label is a fluorophore.

19. The method of claim 18, wherein the fluorophore is FITC, PE, PerCP-Cy5.5 or PE-Cy7.

20. The method of claim 1, wherein the anti-hENT1 antibody is detectably labeled.

21. The method of claim 20, wherein the label is a fluorophore.

22. The method of claim 21, wherein the fluorophore is FITC or PE.

23. The method of claim 1, wherein the anti-hENT1 antibody comprises:
(a) a variable heavy chain complementarity determining region 1 (VH CDR1) sequence comprising the amino acid sequence GYTFTDYE (SEQ ID NO: 10);
(b) a variable heavy chain complementarity determining region 2 (VH CDR2) sequence comprising the amino acid sequence IDPETGAI (SEQ ID NO: 11) or the amino acid sequence IDPETGKT (SEQ ID NO: 40);
(c) a variable heavy chain complementarity determining region 3 (VH CDR3) sequence comprising the amino acid sequence TREFTY (SEQ ID NO: 12) or the amino acid sequence TRELTY (SEQ ID NO: 41);
(d) a variable light chain complementarity determining region 1 (VL CDR1) sequence comprising the amino acid sequence QSLLFSNGKTY (SEQ ID NO: 24);
(e) a variable light chain complementarity determining region 2 (VL CDR2) sequence comprising the amino acid sequence LVS (SEQ ID NO: 25); and
(f) a variable light chain complementarity determining region 3 (VL CDR3) sequence comprising the amino acid sequence VQGTHFPWT (SEQ ID NO: 26).

24. The method of method of claim 1, wherein the anti-hENT1 antibody comprises a heavy chain variable sequence comprising an amino acid sequence selected from SEQ ID NO: 2, 4, 6, 8, 9, 28, 30, 32, 34, 36, 38 and 39 and a light chain variable sequence comprising the amino acid sequence selected from SEQ ID NO: 14, 16, 18, 20, 22, 23, 43, 45, 47 and 49.

25. A method of detecting a level of hENT1 expression in a sample, the method comprising the steps of:
a) contacting a first portion of the sample with one or more antibodies that bind a cell surface marker or cell marker under conditions sufficient to allow binding between the antibody and the cell surface markers;
b) selecting for the cells bound to the one or more antibodies in the first portion of the sample of step a);
c) permeabilizing the selected cells from the first portion of the sample;
d) contacting the permeabilized cells from the first portion of the sample with an antibody that binds hENT1 under conditions sufficient to allow binding between the antibody and hENT1; and
e) detecting the level of cells from the first portion of the sample bound to the anti-hENT1 antibody using flow cytometry.

26. The method of claim 25, wherein the cell surface marker or cell marker is selected from CD45, CD163, CD33, CD34, CD38, CD123, CD117, CD13, CD64, HLA-DR, myeloperoxidase (MPO) and combinations thereof.

27. The method of claim 25, wherein the one or more antibodies that bind a cell surface marker or cell marker is detectably labeled.

28. The method of claim 27, wherein the label is a fluorophore.

29. The method of claim 28, wherein the fluorophore is FITC, PE, PerCP-Cy5.5 or PE-Cy7.

30. The method of claim 25, wherein the anti-hENT1 antibody is detectably labeled.

31. The method of claim 30, wherein the label is a fluorophore.

32. The method of claim 31, wherein the fluorophore is FITC or PE.

33. The method of claim 25, wherein the anti-hENT1 antibody comprises:
(a) a variable heavy chain complementarity determining region 1 (VH CDR1) sequence comprising the amino acid sequence GYTFTDYE (SEQ ID NO: 10);
(b) a variable heavy chain complementarity determining region 2 (VH CDR2) sequence comprising the amino acid sequence IDPETGAI (SEQ ID NO: 11) or the amino acid sequence IDPETGKT (SEQ ID NO: 40);
(c) a variable heavy chain complementarity determining region 3 (VH CDR3) sequence comprising the amino acid sequence TREFTY (SEQ ID NO: 12) or the amino acid sequence TRELTY (SEQ ID NO: 41);
(d) a variable light chain complementarity determining region 1 (VL CDR1) sequence comprising the amino acid sequence QSLLFSNGKTY (SEQ ID NO: 24);
(e) a variable light chain complementarity determining region 2 (VL CDR2) sequence comprising the amino acid sequence LVS (SEQ ID NO: 25); and
(f) a variable light chain complementarity determining region 3 (VL CDR3) sequence comprising the amino acid sequence VQGTHFPWT (SEQ ID NO: 26).

34. The method of claim 25, wherein the anti-hENT1 antibody comprises a heavy chain variable sequence comprising an amino acid sequence selected from SEQ ID NO: 2, 4, 6, 8, 9, 28, 30, 32, 34, 36, 38 and 39 and a light chain variable sequence comprising the amino acid sequence selected from SEQ ID NO: 14, 16, 18, 20, 22, 23, 43, 45, 47 and 49.

35. The method of claim 25, wherein step a) of the method further comprises contacting a second portion of the sample with a solution comprising an immunogenic hENT1 peptide and one or more antibodies that bind a cell surface marker or cell marker under conditions sufficient to allow binding between the antibody and the cell surface markers; wherein step b) further comprises selecting for the cells bound to the one or more antibodies in the second portion of the sample; wherein step c) further comprises permeabilizing the selected cells from the second portion of the sample; wherein step d) further comprises contacting the permeabilized cells from second portion of the sample with an antibody that binds hENT1 under conditions sufficient to allow binding between the antibody and hENT1; and wherein step e) further comprises detecting the level of cells from the second portion of the sample bound to the anti-hENT1 antibody using flow cytometry, and the method further comprising step f) determining the difference between the level of cells from the first portion of the sample bound to the anti-hENT1 antibody and the level of cells from the second portion of the sample bound to the anti-hENT1 antibody, thereby determining the level of hENT1 expression in the sample.

36. A method of detecting a level of hENT1 expression in a subject, the method comprising the steps of:
a) contacting a sample from the subject with one or more antibodies that bind a cell surface marker or cell marker under conditions sufficient to allow binding between the antibody and the cell surface markers, wherein the sample comprises leukemic blast cells, lymphocytes and at least one or more additional types of normal, non-leukemic blood cells selected from monocytes, granulocytes and eosinophils;
b) permeabilizing the cells from the sample;
c) contacting the permeabilized cells from the sample with an antibody that binds hENT1 under conditions sufficient to allow binding between the antibody and hENT1, wherein the anti-hENT1 antibody is detectably labeled with a fluorophore;
d) detecting by flow cytometry the fluorescence level of:
(i) the one or more additional types of normal, non-leukemic blood cells from the sample;
(ii) the lymphocytes from the sample; and
(iii) the leukemic blast cells from the sample;
e) determining a control ratio level of hENT1 expression in the sample by comparing the fluorescence level of each additional type of normal, non-leukemic blood cell in (i) with the fluorescence level of the lymphocytes in (ii);
f) determining the ratio level of hENT1 expression in the leukemic blast cells from the sample by comparing the fluoresce level of the leukemic blast cells in (iii) with the fluorescence level of the lymphocytes in (ii); and
g) comparing the ratio level of step f) with the control ratio level of step e) to determine the level of hENT1 expression in the subject.

37. The method of claim 36, wherein the subject is suffering from acute myeloid leukemia (AML).

38. The method of claim 36, wherein the hematological disorder is myelodysplastic syndrome (MDS), acute lymphoblastic leukemia (ALL) or chronic myelomonocytic leukemia (CMML),

39. The method of claim 36, wherein the cell surface marker or cell marker is selected from CD45, CD163, CD33, CD34, CD38, CD123, CD117, CD13, CD64, HLA-DR, myeloperoxidase (MPO) and combinations thereof.

40. The method of claim 36, wherein the one or more antibodies that bind a cell surface marker or cell marker is detectably labeled.

41. The method of claim 40, wherein the label is a fluorophore.

42. The method of claim 41, wherein the fluorophore is FITC, PE, PerCP-Cy5.5 or PE-Cy7.

43. The method of claim 36, wherein the detectably labeled anti-hENT1 antibody comprises a fluorophore selected from FITC and PE.

44. The method of claim 36, wherein the anti-hENT1 antibody comprises:
(a) a variable heavy chain complementarity determining region 1 (VH CDR1) sequence comprising the amino acid sequence GYTFTDYE (SEQ ID NO: 10);
(b) a variable heavy chain complementarity determining region 2 (VH CDR2) sequence comprising the amino acid sequence IDPETGAI (SEQ ID NO: 11) or the amino acid sequence IDPETGKT (SEQ ID NO: 40);
(c) a variable heavy chain complementarity determining region 3 (VH CDR3) sequence comprising the amino acid sequence TREFTY (SEQ ID NO: 12) or the amino acid sequence TRELTY (SEQ ID NO: 41);
(d) a variable light chain complementarity determining region 1 (VL CDR1) sequence comprising the amino acid sequence QSLLFSNGKTY (SEQ ID NO: 24);
(e) a variable light chain complementarity determining region 2 (VL CDR2) sequence comprising the amino acid sequence LVS (SEQ ID NO: 25); and
(f) a variable light chain complementarity determining region 3 (VL CDR3) sequence comprising the amino acid sequence VQGTHFPWT (SEQ ID NO: 26).

45. The method of claim 36, wherein the anti-hENT1 antibody comprises a heavy chain variable sequence comprising an amino acid sequence selected from SEQ ID NO: 2, 4, 6, 8, 9, 36, 30, 32, 34, 36, 38 and 39, and a light chain variable sequence comprising the amino acid sequence selected from SEQ ID NO: 14, 16, 18, 20, 22, 23, 43, 45, 47 and 49.
